SEQUENCE LISTING (54 sequences)

SEQ ID NO: 1 - protein, 554 aa, Parvovirus B19
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 2 - protein, 5 aa, Artificial Sequence
  19 VH CDR 1
SYTMY

SEQ ID NO: 3 - protein, 17 aa, Artificial Sequence
  3 - VH CDR2
YVNPSSGYTNYNQKFKD

SEQ ID NO: 4 - protein, 14 aa, Artificial Sequence
  4 - 19 VH CDR3
HGNYRYDGYYGMDC

SEQ ID NO: 5 - protein, 5 aa, Artificial Sequence
  5 - 25 VH CDR1
NYWMH

SEQ ID NO: 6 - protein, 17 aa, Artificial Sequence
  6 - 25 VH CDR2
RIDPYDSETHYNQKFKD

SEQ ID NO: 7 - protein, 13 aa, Artificial Sequence
  7 - 25VH CDR3
YYYGSNYVWHFDV

SEQ ID NO: 8 - protein, 5 aa, Artificial Sequence
  8 - 61 VH CDR1
SYTMH

SEQ ID NO: 9 - protein, 17 aa, Artificial Sequence
  9- 61 VH CDR2
YINPSSGYTNYNQKFKD

SEQ ID NO: 10 - protein, 10 aa, Artificial Sequence
  10 - 61 VH CDR3
PDGYYAWFAY

SEQ ID NO: 11 - protein, 5 aa, Artificial Sequence
  11 - 91 VH CDR1
DYFMN

SEQ ID NO: 12 - protein, 17 aa, Artificial Sequence
  12 - 91 VH CDR2
RINPYNGDSFYNQNFRG

SEQ ID NO: 13 - protein, 10 aa, Artificial Sequence
  13 - 91 VH CDR3
YRYDGYAMDY

SEQ ID NO: 14 - protein, 11 aa, Artificial Sequence
  14 - 19 VL CDR1
KASEDIYNRLA

SEQ ID NO: 15 - protein, 7 aa, Artificial Sequence
  15 - 19 VL CDR2
GATSLET

SEQ ID NO: 16 - protein, 9 aa, Artificial Sequence
  16 - 19 VL CDR3
QQYWSTPYT

SEQ ID NO: 17 - protein, 16 aa, Artificial Sequence
  17 - 25 VL CDR1
KSSQSLLDSDGRTYLN

SEQ ID NO: 18 - protein, 7 aa, Artificial Sequence
  18 - 25 VL CDR2
LVSKLDS

SEQ ID NO: 19 - protein, 9 aa, Artificial Sequence
  19 - 25 VL CDR3
WQGTHLPYT

SEQ ID NO: 20 - protein, 11 aa, Artificial Sequence
  20 - 61 VL CDR1
KASEDIYNRLA

SEQ ID NO: 21 - protein, 7 aa, Artificial Sequence
  21 - 61 VL CDR2
GATSLET

SEQ ID NO: 22 - protein, 8 aa, Artificial Sequence
  22 - 61 VL CDR3
QQYWSTPT

SEQ ID NO: 23 - protein, 16 aa, Artificial Sequence
  23 - 91 VL CDR1
RSSQSLVHSDGNTYLH

SEQ ID NO: 24 - protein, 7 aa, Artificial Sequence
  24 - 91 VL CDR2
KVSNRFS

SEQ ID NO: 25 - protein, 9 aa, Artificial Sequence
  25 - 91 VL CDR3
SQSTHVPFT

SEQ ID NO: 26 - protein, 123 aa, Artificial Sequence
  26 - 19 VH
QVQLQQSGAELARPGASVKMSCKASGYTFTSYTMYWVKQRPGQGLEWIGYVNPSSGYTNYNQKFKDKATLTADKSSSTAYMQLSSLTSEDSAVYYCARHGNYRYDGYYGMDCWGQGTSVTVSS

SEQ ID NO: 27 - protein, 122 aa, Artificial Sequence
  27 - 25 VH
QVQLQQPGAELVRPGASVKLSCKASGYTFTNYWMHWVKQRPEQGLEWIGRIDPYDSETHYNQKFKDKAILTVDTSSNTAYMQLSSLTSEDSAVYYCTSYYYGSNYVWHFDVWGAGTTVTVSS

SEQ ID NO: 28 - protein, 119 aa, Artificial Sequence
  28 - 61 VH
QVQLQQSGAELARPGASVKMSCKASGYTFTSYTMHWVKQRPGQGLEWIGYINPSSGYTNYNQKFKDKATLTADKSSSTAYMQLSSLTSEDSAVYYCARPDGYYAWFAYWGQGTLVTVSA

SEQ ID NO: 29 - protein, 119 aa, Artificial Sequence
  29 - 91 VH
EVQLQQSGPELVKPGASVKISCKASGYSFTDYFMNWVKQSHGKSLEWIGRINPYNGDSFYNQNFRGSVTLTVDKSSGTAHMELLSLTSEDSTVYYCGAYRYDGYAMDYWGQGTSVTVSS

SEQ ID NO: 30 - protein, 107 aa, Artificial Sequence
  30 - 19 VL
DIQMTQSSSSFSVSLGDRVTITCKASEDIYNRLAWYQQKPGNAPRLLISGATSLETGVPSRFSGSGSGKDYTLSITSLQTEDVATYYCQQYWSTPYTFGGGTKLEIK

SEQ ID NO: 31 - protein, 112 aa, Artificial Sequence
  31 - 25 VL
DVVMTQTPLTLSVTIGQPASISCKSSQSLLDSDGRTYLNWLLQRPGQSPKRLIYLVSKLDSGVPDRFTGSGSGTDFTLKISRVEAEDLGIYYCWQGTHLPYTFGGGTKLEIK

SEQ ID NO: 32 - protein, 106 aa, Artificial Sequence
  32 - 61 VL
DIQMTQSSSSFSVSLGDRVTITCKASEDIYNRLAWYQQKPGNAPRLLISGATSLETGVPSRFSGSGSGKDYTLSITSLQTEDVATYYCQQYWSTPTFGGGTKLEIK

SEQ ID NO: 33 - protein, 112 aa, Artificial Sequence
  33 - 91 VL
DVVMTQTPLSLPVSLGDQASISCRSSQSLVHSDGNTYLHWYLQKPGQSPKLLIYKVSNRFSGVPDRFSGSGSGTDFTLKISRVEAEDLGVYFCSQSTHVPFTFGSGTKLEIK

SEQ ID NO: 34 - DNA, 369 bp, Artificial Sequence
  34 - 19 VH
caggtccagctgcagcagtctggggctgaactggcaagacctggggcctcagtgaagatgtcctgcaaggcttctggctacacctttactagctacacgatgtactgggtaaaacagaggcctggacagggtctggagtggattggatacgttaatcctagtagtggttatactaattacaatcagaagttcaaggacaaggccacattgactgcagacaaatcctccagcacagcctacatgcaactgagcagcctgacatctgaggactctgcagtctattactgtgcaagacatgggaactataggtacgacgggtactatggtatggactgctggggtcaaggaacctcagtcaccgtctcctca

SEQ ID NO: 35 - DNA, 365 bp, Artificial Sequence
  35 - 25 VH
caggtccaactgcagcagcctggggctgagctggtgaggcctggggcttcagtgaagctgtcctgcaaggcttctggctacacgttcaccaactactggatgcactgggttaaacagaggcctgagcaaggccttgagtggattggaaggattgatccttacgatagtgaaactcactacaatcaaaagttcaaggataaggccatattgactgtggacacatccccaacacagcctacatgcaactcagcagcctgacatctgaggactctgcggtctattactgtacatcgtattactacggtagtaattacgtttggcacttcgatgtctggggcgcagggaccacggtcaccgtctcctca

SEQ ID NO: 36 - DNA, 357 bp, Artificial Sequence
  36 - 61 VH
caggtccagctgcagcagtctggggctgaactggcaagacctggggcctcagtgaagatgtcctgcaaggcttctggctacacctttactagctacacgatgcactgggtaaaacagaggcctggacagggtctggaatggattggatacattaatcctagcagtggttatactaattacaatcagaagttcaaggacaaggccacattgactgcagacaaatcctccagcacagcctacatgcaactgagcagcctgacatctgaggactctgcagtctattactgtgcaagaccagatggttactacgcctggtttgcttactggggccaagggactctggtcactgtctctgca

SEQ ID NO: 37 - DNA, 357 bp, Artificial Sequence
  37 - 91 VH
gaggttcagctgcagcagtctggacctgagctggtgaagcctggggcttcagtgaagatttcctgcaaggcttctggttactcatttactgactactttatgaactgggtgaagcagagccatggaaagagccttgagtggattggacgtattaatccttacaatggtgatagtttctacaaccagaacttcaggggcagtgtcacattgactgttgacaaatcctctggcacagcccacatggagctcctgagcctgacatctgaggactctacagtctattattgtggagcctataggtacgacgggtatgctatggactactggggtcaaggaacctcagtcaccgtctcctca

SEQ ID NO: 38 - DNA, 321 bp, Artificial Sequence
  38 - 19 VL
gacatccagatgacacaatcttcatcctccttttctgtatctctaggagacagagtcaccattacttgcaaggcaagtgaggacatatataatcggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtactccgtacacgttcggaggggggaccaagctggaaataaaa

SEQ ID NO: 39 - DNA, 336 bp, Artificial Sequence
  39 - 25 VL
gatgttgtgatgacccagactcctctcactttgtcggttaccattggacaaccagcctccatctcttgtaagtcaagtcagagcctcttagatagtgatggaaggacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggaatttattattgctggcaaggtacacatcttccttacacgttcggaggggggaccaagctggaaataaaa

SEQ ID NO: 40 - DNA, 318 bp, Artificial Sequence
  40 - 61 VL
gacatccagatgacacaatcttcatcctccttttctgtatctctaggagacagagtcaccattacttgcaaggcaagtgaggacatatataatcggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgctacttattactgtcaacagtattggagtactccgacgttcggtggaggcaccaagctggaaatcaaa

SEQ ID NO: 41 - DNA, 336 bp, Artificial Sequence
  41 - 91 VL
gatgttgtgatgacccaaactccactctccctgcctgtcagtcttggagatcaagcctccatctcttgcagatctagtcagagccttgtacacagtgatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttccattcacgttcggctcggggacaaagttggaaataaaa

SEQ ID NO: 42 - protein, 554 aa, Artificial Sequence
  Construct A - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQFTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 43 - protein, 554 aa, Artificial Sequence
  Construct B - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQWTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 44 - protein, 554 aa, Artificial Sequence
  Construct C - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQATDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 45 - protein, 554 aa, Artificial Sequence
  Construct D - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYAAGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 46 - protein, 554 aa, Artificial Sequence
  Construct E - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYNNGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 47 - protein, 554 aa, Artificial Sequence
  Construct F - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTNNFTDTIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 48 - protein, 554 aa, Artificial Sequence
  Construct G - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTNNYTDTIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 49 - protein, 554 aa, Artificial Sequence
  Construct H - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTAFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 50 - protein, 554 aa, Artificial Sequence
  Construct I - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTFFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 51 - protein, 554 aa, Artificial Sequence
  Construct J - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTNNFTDNIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 52 - protein, 554 aa, Artificial Sequence
  Construct K - polypeptide
MTSVNSAEASTGAGGGGSNPVKSMWSEGATFSANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFQLLGTGGTATMSYKFPPVPPENLEGCSQHFYEMYNPLYGSRLGVPDTLGGDPKFRSLTHEDHAIQPQNFMPGPLVNSVSTKEGDSSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTNNYTDNIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 53 - protein, 554 aa, Artificial Sequence
  Genotype 2 - VP2  -  AAK95572.2
MTSVNSAEASTGAGGGGSNPVKSMWSEGATFTANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGDVNTQGISGDSKKLASEESAFYVLEHSSFELLGTGGSATMSYKFPPVPPENLEGCSQHFYEMYNPLYGSRLGVPDTLGGDPKFRSLTHEDHAIQPQNFMPGPLVNSVSTKEGDTSNTGAGKALTGLSTGTSQSTRISLRPGPVSQPYHHWDTDKYVTGINAISHGQTTYGNAEDKEYQQGVGRFPNEKEQLKQLQGLNIHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

SEQ ID NO: 54 - protein, 554 aa, Artificial Sequence
  Genotype 3 VP2 - CAC80622.1
MTSVNSAEASTGAGGGGSNPTKSMWSEGATFTANSVTCTFSRQFLIPYDPEHHYKVFSPAASSCHNASGKEAKVCTISPIMGYSTPWRYLDFNALNLFFSPLEFQHLIENYGSIAPDALTVTISEIAVKDVTDKTGGGVQVTDSTTGRLCMLVDHEYKYPYVLGQGQDTLAPELPIWVYFPPQYAYLTVGEVNTQGISGDSKKLASEESAFYVLEHSSFELLGTGGSATMSYKFPAVPPENLEGCSQHFYEMYNPLYGSRLGVPDTLGGDPKFRSLTHEDHAIQPQNFMPGPLINSVSTKEGDNSNTGAGKALTGLSTGTSQNTRISLRPGPVSQPYHHWDTDKYVTGINAISHGQTTYGNAEDKEYQQGVGRFPNEKEQLKQLQGLNMHTYFPNKGTQQYTDQIERPLMVGSVWNRRALHYESQLWSKIPNLDDSFKTQFAALGGWGLHQPPPQIFLKILPQSGPIGGIKSMGITTLVQYAVGIMTVTMTFKLGPRKATGRWNPQPGVYPPHAAGHLPYVLYDPTATDAKQHHRHGYEKPEELWTAKSRVHPL

(19) United States
(12) Patent Application Publication
(10) Pub. No.: US 2023/0049732 A1
(43) Pub. Date: Feb. 16, 2023

(54) VIRUS-LIKE PARTICLE BINDING AGENTS, RELATED COMPOSITIONS, AND RELATED METHODS

(71) Applicant: UNIVERSITY OF LOUISVILLE RESEARCH FOUNDATION, INC., Louisville, KY (US)

(72) Inventors: Shin-je GHIM, Louisville, KY (US); A. Bennett JENSON, Louisville, KY (US); Maryam ZAHIN, Louisville, KY (US)

(73) Assignee: UNIVERSITY OF LOUISVILLE RESEARCH FOUNDATION, INC., Louisville, KY (US)

(21) Appl. No.: 17/757,599

(22) PCT Filed: Dec. 30, 2020
(86) PCT No.: PCT/US2020/067394

§ 371 Date: Jun. 17, 2022

Related U.S. Application Data
(60) Provisional application No. 62/954,716, filed on Dec. 30, 2019.

(51) Int. Cl. C07K 16/08; G01N 33/569; G01N 33/533

(52) U.S. Cl. CPC C07K 16/081; G01N 33/56983; G01N 33/533; C07K 2317/33; C12N 2750/14034; G01N 2333/015

(57) ABSTRACT

Some embodiments of the invention include virus-like particle (VLP) binding agents, and related polynucleotides, cells, methods of making, and compositions. Other embodiments of the invention include methods of detecting VLPs, parvovirus, erythrovirus or parvovirus B19 using a VLP binding agent and diagnostic methods for parvovirus, erythrovirus or parvovirus B19. Further embodiments include methods for administering VLP binding agents to an animal. Other embodiments include treating parvovirus, erythrovirus or parvovirus B19 infections and other diseases. Additional embodiments of the invention are also discussed.

### CROSS-REFERENCE TO RELATED APPLICATIONS

This application claims the benefit of U.S. Provisional Application No. 62/954,716, filed Dec. 30, 2019 entitled “Antibodies Against Virus-Like-Particles and Mutant Virus-Like-Particles, Related Compositions, and Related Methods” which is herein incorporated by reference in its entirety.

### REFERENCE TO A SEQUENCE LISTING

The instant application contains a Sequence Listing that has been submitted in ASCII format via EFS-Web and is hereby incorporated by reference in its entirety. Said ASCII copy, created on Dec. 18, 2020, is named 35783_04159_PCT1_December_2020_ST25.txt and is 85 KB in size.

### BACKGROUND

Parvovirus is the common name used to refer to all of the viruses in the Parvoviridae family Erythrovirus is a genus of the Parvoviridae family containing viruses that infect erythrocyte progenitor cells. Parvoviruses can infect many animals (e.g., mammals, porcine, canine, feline, primates, monkeys, and humans). Parvovirus B19 belongs to erythrovirus and contains three genotypes (Servant-Delmas et al., J Virol. (October 2010) Vol. 84, No. 19, pp. 9658-9665). Genotype 1 consists of prototype parvovirus B19. Genotype 2 includes the Lali strain and the A6 strain, genotype 3a the V9 strain, and genotype 3b the D91.1 strain. The clinical spectrum associated with genotype 1 or 2 or 3 virus infection can be similar.

Parvovirus B19 (a species of the erythrovirus genus) can cause severe and sometimes fatal diseases in fetuses and newborns, such as hydrops fetalis, intrauterine fetal death and erythema infectiosum (fifth disease) in children. Older children and adults with either hereditary diseases (e.g., sickle cell anemia or Thalassemia) or acquired diseases (e.g., malaria or anemia) are at risk for developing parvovirus B19-induced red cell aplasia or death. Chronic anemia in immunodeficient, organ transplant, or HIV patients has contributed to parvovirus B19 infection. A cellular receptor for parvovirus B19 is the blood group P antigen, a globoside, that is expressed in erythroid precursors and maintained on mature red blood cells (RBCs). To date, no vaccine is available to prevent human erythrovirus, including parvovirus B19 infection. Accordingly, some embodiments of the present invention include virus-like particle (VLP) binding agents (e.g., antibodies and monoclonal antibodies) that have many uses including but not limited to detecting VLPs, parvovirus, erythrovirus, and parvovirus B19, diagnosing parvovirus, erythrovirus, and parvovirus B19, and treating parvovirus, erythrovirus, and parvovirus B19.

Some embodiments of the invention address one or more of the above issues. Some embodiments of the invention include virus-like particle (VLP) binding agents, and related polynucleotides, cells, methods of making, and compositions. Other embodiments of the invention include methods of detecting VLPs, parvovirus, erythrovirus or parvovirus B19 using a VLP binding agent and diagnostic methods for parvovirus, erythrovirus or parvovirus B19. Further embodiments include methods for administering VLP binding agents to an animal. Other embodiments include treating parvovirus, erythrovirus or parvovirus B19 infections and other diseases. Additional embodiments of the invention are also discussed.

### SUMMARY

Some embodiments of the present invention include a VLP binding agent that specifically binds to a VLP, a parvovirus, an erythrovirus, or a parvovirus B19. In other embodiments, the VLP is (a) a wtVLP, (b) an mVLP comprising a polypeptide comprising a VP2 polypeptide with at least one amino acid modification relative to a wild type VP2, or (c) both. In still other embodiments, the wild type VP2 has the amino acid sequence of SEQ ID NO: 1, and the at least one amino acid modification comprises a substitution at Y401, a substitution at Q399, a substitution at Q400, a substitution at Q404, a substitution at Q368, a substitution at Q369, a substitution at Y392, Y401F, Y401W, Y401A, Q368A, Q369A, Q368N, Q369N, Q399N, Q400N, Q404T, Y392A, Y392F, Q404N, Y401P, T402A, D403A, Q404A, or combinations thereof. In other embodiments, the at least one amino acid modification comprises one or more of Y401F, Y401W, Q368A, Q369A, Q399N, Q400N, or Q404T. In yet other embodiments, the VP2 polypeptide is construct A, construct B, construct D, or construct F. In certain embodiments, the VLP binding agent is an antibody, a monoclonal antibody, an antigen binding fragment, or an antibody fragment. In some embodiments, the VLP binding agent comprises an amino acid sequence with (a) SEQ ID NOS:2-4 and 14-16, each with up to four conservative amino acid substitutions, (b) SEQ ID NOS:5-7 and 17-19, each with up to four conservative amino acid substitutions, (c) SEQ ID NOS:8-10 and 20-22, each with up to four conservative amino acid substitutions, or (d) SEQ ID NOS:11-13 and 23-25, each with up to four conservative amino acid substitutions. In certain embodiments, the VLP binding agent comprises an amino acid sequence with (a) SEQ ID NOS:2-4 and 14-16, (b) SEQ ID NOS:5-7 and 17-19, (c) SEQ ID NOS:8-10 and 20-22, or (d) SEQ ID NOS:11-13 and 23-25. In other embodiments, the VLP binding agent comprises an amino acid sequence with (a) at least about 90% sequence identity to any of SEQ ID NOs:26-29; and/or (b) at least about 90% sequence identity to any of SEQ ID NOs:30-33. In still other embodiments, the VLP binding agent comprises an amino acid sequence with (a) at least one of SEQ ID NOs:26-29; and/or (b) at least one of SEQ ID NOs:30-33. In yet other embodiments, the VLP binding agent comprises an amino acid sequence with (a) SEQ ID NOs:26 and 30, (b) SEQ ID NOs:27 and 31, (c) SEQ ID NOs:28 and 32, or (d) SEQ ID NOs:29 and 33. In certain embodiments, the VLP binding agent is detectably labeled.

Some embodiments of the present invention include a cell for producing the VLP binding agent of any disclosed herein. Some embodiments of the present invention include a method for making the VLP binding agent of any disclosed herein comprising (a) culturing the cell of any disclosed herein, and (b) isolating the VLP binding agent. Some embodiments of the present invention include a composition comprising the VLP binding agent of any disclosed herein. Some embodiments of the present invention include a pharmaceutical composition comprising the VLP binding agent of any disclosed herein. Some embodiments of the present invention include a polynucleotide comprising a polynucleotide that encodes the VLP binding agent of any disclosed herein.

Some embodiments of the present invention include a method of detecting parvovirus, erythrovirus, parvovirus B19, or a VLP in a sample comprising contacting the sample with the VLP binding agent of any disclosed herein, the composition of any disclosed herein, or the pharmaceutical composition of any disclosed herein. In certain embodiments, the VLP binding agent is detectably labeled. In other embodiments, the label is selected from the group consisting of immunofluorescent label, chemiluminescent label, phosphorescent label, enzyme label, radiolabel, avidin/biotin, colloidal gold particles, colored particles and magnetic particles. In still other embodiments, the detecting is determined by radioimmunoassay, Western blot assay, cytometry, immunofluorescent assay, enzyme immunoassay, ELISA, immunoprecipitation assay, chemiluminescent assay, or immunohistochemical assay. In yet other embodiments, the detecting is of (a) parvovirus, (b) parvovirus B19, (c) the wtVLP made from a VP2 polypeptide of SEQ ID NO:1 or (d) construct F.

Some embodiments of the present invention include a method for diagnosis in an animal with a parvovirus infection, an erythrovirus infection, or a parvovirus B19 infection, the method comprising (a) detecting whether parvovirus, erythrovirus, or parvovirus B19 is in a sample from the animal, comprising the method of detecting according to any detection method disclosed herein, and (b) diagnosing the animal with a parvovirus infection, an erythrovirus infection, or a parvovirus B19 infection, if the presence of parvovirus, erythrovirus, or parvovirus B19 in the sample is detected. In other embodiments, the method is for diagnosis for a parvovirus infection or a parvovirus B19 infection. In certain embodiments, the animal is a mammal. In still other embodiments, the animal is a human or a primate.

Some embodiments of the present invention include a method for treating an animal for a parvovirus infection, a disease related to a parvovirus infection, an erythrovirus infection, a disease related to an erythrovirus infection, a parvovirus B19 infection, or a disease related to a parvovirus B19 infection, comprising one or more administrations of one or more compositions comprising one or more VLP binding agents of any disclosed herein. In certain embodiments, at least one of the one or more compositions does not comprise an adjuvant. In some embodiments, at least one of the one or more compositions further comprises a carrier or an adjuvant. In still other embodiments, at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, ADJUPLEX™ vaccine adjuvant, MF59, TITERMAX®, or combinations thereof. In yet other embodiments, at least one of the one or more the compositions comprises a pharmaceutical composition. In certain embodiments, at least one of the one or more administrations comprises parenteral administration, a mucosal administration, intravenous administration, subcutaneous administration, topical administration, intradermal administration, oral administration, sublingual administration, intranasal administration, or intramuscular administration. In some embodiments, if there is more than one administration at least one composition used for at least one administration is different from the composition of at least one other administration. In other embodiments, the animal is a human or a primate. In still other embodiments, the animal is in need of the treatment. In yet other embodiments, the method is for treating an erythrovirus infection, a disease related to an erythrovirus infection, a parvovirus B19 infection, or a disease related to a parvovirus B19 infection. In other embodiments, the method is for treating an erythrovirus infection, a parvovirus B19 infection, a disease related to an erythrovirus infection, a disease related to parvovirus B19 infection, hydrops fetalis intrauterine fetal death, erythema infectiosum (i.e., fifth disease), sickle cell anemia, Thalassemia, anemia, anemia induced by malaria, parvovirus B19-induced red cell aplasia (TRCA), chronic anemia, acute arthropathy, persistent arthropathy, aplastic crisis, arthritis, hepatitis, myocarditis, hepatosplenomegaly, systemic lupus erythematosus, meningiencephalitis, or fibromyalgia. In certain embodiments, the method induces an immune response, is a therapeutic treatment, or is a combination thereof.

Some embodiments of the present invention include a method for inducing an immune response in an animal, comprising one or more administrations of one or more compositions comprising one or more VLP binding agents of any disclosed herein. In certain embodiments, at least one of the one or more compositions does not comprise an adjuvant. In other embodiments, at least one of the one or more compositions further comprises a carrier or an adjuvant. In some embodiments, at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, ADJUPLEX™ vaccine adjuvant, MF59, TITERMAX®, or combinations thereof. In still other embodiments, at least one of the one or more the compositions comprises a pharmaceutical composition. In yet other embodiments, at least one of the one or more administrations comprises parenteral administration, a mucosal administration, intravenous administration, subcutaneous administration, topical administration, intradermal administration, oral administration, sublingual administration, intranasal administration, or intramuscular administration. In certain embodiments, if there is more than one administration at least one composition used for at least one administration is different from the composition of at least one other administration. In other embodiments, the animal is a human or a primate. In some embodiments, the animal is in need of the treatment. In yet other embodiments, the method is for treating an erythrovirus infection, a disease related to an erythrovirus infection, a parvovirus B19 infection, or a disease related to a parvovirus B19 infection. In still other embodiments, the method is for treating an erythrovirus infection, a parvovirus B19 infection, a disease related to an erythrovirus infection, a disease related to parvovirus B19 infection, hydrops fetalis intrauterine fetal death, erythema infectiosum (i.e., fifth disease), sickle cell anemia, Thalassemia, anemia, anemia induced by malaria, parvovirus B19-induced red cell aplasia (TRCA), chronic anemia, acute arthropathy, persistent arthropathy, aplastic crisis, arthritis, hepatitis, myocarditis, hepatosplenomegaly, systemic lupus erythematosus, meningiencephalitis, or fibromyalgia. In other embodiments, the method induces an immune response, is a therapeutic treatment, or is a combination thereof.

Some embodiments of the present invention include a method for treating an animal for a parvovirus infection, a disease related to a parvovirus infection, an erythrovirus infection, a disease related to an erythrovirus infection, a parvovirus B19 infection, or a disease related to a parvovirus B19 infection, comprising (a) detecting whether parvovirus, erythrovirus, or parvovirus B19 is in a sample from the animal, comprising the method of detecting according to any detection method disclosed hereon, and (b) administering one or more administrations of one or more compositions comprising one or more of an mVLP, a VLP binding agent of any disclosed herein, or an antibiotic, if the presence of parvovirus, erythrovirus, or parvovirus B19 in the sample is detected. In certain embodiments, the antibiotic comprises ampicillin, a cephalexin, or a flouroquinolone, or combinations thereof. In other embodiments, (a) the mVLP comprises a VP2 polypeptide with at least one amino acid modification relative to a wild type VP2 and (b) the wild type VP2 has the amino acid sequence of SEQ ID NO: 1. In yet other embodiments, (a) the mVLP comprises a VP2 polypeptide with at least one amino acid modification relative to a wild type VP2, (b) the wild type VP2 has the amino acid sequence of SEQ ID NO: 1, (c) the at least one amino acid modification (1) comprises (i) Y401F and (ii) Q399N or Q404T, (2) is Y401F, (3) is Q368A and Q369A, (4) is Q399N, Q400N, and Q404T, or (5) is Y392A, and (d) the VP2 polypeptide is not construct J. In still other embodiments, the VP2 polypeptide sequence has at least about 90% identity to SEQ ID NO: 1. In certain embodiments, the VP2 polypeptide sequence has at least about 95% identity to SEQ ID NO: 1. In some embodiments, the at least one amino acid modification comprises (a) Y401F and (b) Q399N or Q404T. In other embodiments, the VP2 polypeptide is selected from the group consisting of construct A, construct D, construct F, construct G, and construct H. In some embodiments, the VP2 polypeptide is Construct F. In yet other embodiments, the mVLP comprises a VP2 that has at least one amino acid modification (1) comprising (a) Y401F and (b) Q399N or Q404T or (2) is Y401F, and the VP2 polypeptide is not construct J. In still other embodiments, at least one of the one or more compositions does not comprise an adjuvant. In other embodiments, at least one of the one or more compositions further comprises a carrier or an adjuvant. In certain embodiments, at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, ADJUPLEX™ vaccine adjuvant, MF59, TITERMAX®, or combinations thereof. In some embodiments, at least one of the one or more the compositions comprises a pharmaceutical composition. In still other embodiments, at least one of the one or more administrations comprises parenteral administration, a mucosal administration, intravenous administration, subcutaneous administration, topical administration, intradermal administration, oral administration, sublingual administration, intranasal administration, or intramuscular administration. In certain embodiments, if there is more than one administration at least one composition used for at least one administration is different from the composition of at least one other administration. In other embodiments, the mVLP of at least one of the one or more compositions is administered to the animal in an amount of from about 0.01 mg of mVLP/kg animal body weight to about 15 mg of mVLP/kg animal body weight. In still other embodiments, the animal is a human or a primate. In yet other embodiments, the animal is in need of the treatment. In some embodiments, the method is for treating an erythrovirus infection, a disease related to an erythrovirus infection, a parvovirus B19 infection, or a disease related to a parvovirus B19 infection. In certain embodiments, the method is for treating an erythrovirus infection, a parvovirus B19 infection, a disease related to an erythrovirus infection, a disease related to parvovirus B19 infection, hydrops fetalis intrauterine fetal death, erythema infectiosum (i.e., fifth disease), sickle cell anemia, Thalassemia, anemia, anemia induced by malaria, parvovirus B19-induced red cell aplasia (TRCA), chronic anemia, acute arthropathy, persistent arthropathy, aplastic crisis, arthritis, hepatitis, myocarditis, hepatosplenomegaly, systemic lupus erythematosus, meningiencephalitis, or fibromyalgia. In yet other embodiments, the method induces an immune response, is a therapeutic treatment, or is a combination thereof.

Some embodiments of the present invention include a method for inducing an immune response in an animal comprising (a) detecting whether parvovirus, erythrovirus, or parvovirus B19 is in a sample from the animal, comprising the method of detecting according to any of claims 18-22, and (b) administering one or more administrations of one or more compositions comprising one or more of an mVLP, a VLP binding agent of any disclosed herein, or an antibiotic, if the presence of parvovirus, erythrovirus, or parvovirus B19 in the sample is detected. In certain embodiments, the antibiotic comprises ampicillin, a cephalexin, or a flouroquinolone, or combinations thereof. In other embodiments, (a) the mVLP comprises a VP2 polypeptide with at least one amino acid modification relative to a wild type VP2 and (b) the wild type VP2 has the amino acid sequence of SEQ ID NO: 1. In yet other embodiments, (a) the mVLP comprises a VP2 polypeptide with at least one amino acid modification relative to a wild type VP2, (b) the wild type VP2 has the amino acid sequence of SEQ ID NO: 1, (c) the at least one amino acid modification (1) comprises (i) Y401F and (ii) Q399N or Q404T, (2) is Y401F, (3) is Q368A and Q369A, (4) is Q399N, Q400N, and Q404T, or (5) is Y392A, and (d) the VP2 polypeptide is not construct J. In other embodiments, the VP2 polypeptide sequence has at least about 90% identity to SEQ ID NO: 1. In still other embodiments, the VP2 polypeptide sequence has at least about 95% identity to SEQ ID NO: 1. In other embodiments, the at least one amino acid modification comprises (a) Y401F and (b) Q399N or Q404T. In certain embodiments, the VP2 polypeptide is selected from the group consisting of construct A, construct D, construct F, construct G, and construct H. In some embodiments, the VP2 polypeptide is Construct F. In still other embodiments, at least one of the one or more compositions does not comprise an adjuvant. In certain embodiments, at least one of the one or more compositions further comprises a carrier or an adjuvant. In some embodiments, if there is more than one administration at least one composition used for at least one administration is different from the composition of at least one other administration. In other embodiments, the animal is a human or a primate. In still other embodiments, at least one of the one or more compositions does not comprise an adjuvant. In yet other embodiments, at least one of the one or more compositions further comprises a carrier or an adjuvant. In certain embodiments, at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, ADJUPLEX™ vaccine adjuvant, MF59, TITERMAX®, or combinations thereof. In certain embodiments, at least one of the one or more the compositions comprises a pharmaceutical composition. In some embodiments, wherein at least one of the one or more administrations comprises parenteral administration, a mucosal administration, intravenous administration, subcutaneous administration, topical administration, intradermal administration, oral administration, sublingual administration, intranasal administration, or intramuscular administration.

Some embodiments of the invention include a method for providing an animal with a VLP binding agent comprising one or more administrations of one or more compositions comprising the VLP binding agent of any disclosed herein, wherein the compositions may be the same or different if there is more than one administration. In certain embodiments, at least one of the one or more compositions does not comprise an adjuvant. In other embodiments, at least one of the one or more compositions further comprises a carrier or an adjuvant. In some embodiments, at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, adjuplex vaccine adjuvant, MF59, titermax, or combinations thereof. In certain embodiments, at least one of the one or more compositions comprises the composition of any disclosed herein or the pharmaceutical composition of any disclosed herein. In still other embodiments, at least one of the one or more administrations comprises parenteral administration, a mucosal administration, intravenous administration, subcutaneous administration, topical administration, intradermal administration, oral administration, sublingual administration, intranasal administration, or intramuscular administration. In yet other embodiments, if there is more than one administration at least one composition used for at least one administration is different from the composition of at least one other administration. In some embodiments, the animal is a human or a primate. In certain embodiments, at least one of the one or more compositions further comprises a VLP (e.g., vtVLP or mVLP) of any of disclosed herein. In other embodiments, at least one of the one or more compositions further comprises an antibiotic. In yet other embodiments, at least one of the one or more compositions further comprises an antibiotic and the antibiotic comprises ampicillin, a cephalexin, or a flouroquinolone, or combinations thereof.

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: Immunoblots of recombinant VLPs of three genotypes of parvovirus B19 (B19V) and two mutated (F₄₀₁—and N₃₉₉N₄₀₀F₄₀₁TDT₄₀₄) parvovirus B19. The VP2 proteins, which were marked with arrows, were examined for reactivity with rabbit polyclonal antibody (PAB) raised against wtVLPs of genotype 1 and MAB8293. The arrows indicate the target VP2 protein band at approximately 58 kDa. Genotypes 1, 2 and 3 (lanes 1, 2 and 3) were not mutated. F₄₀₁-VLPs and N₃₉₉N₄₀₀F₄₀₁TDT₄₀₄-VLPs were composed of VP2 that were mutated from genotype 1.

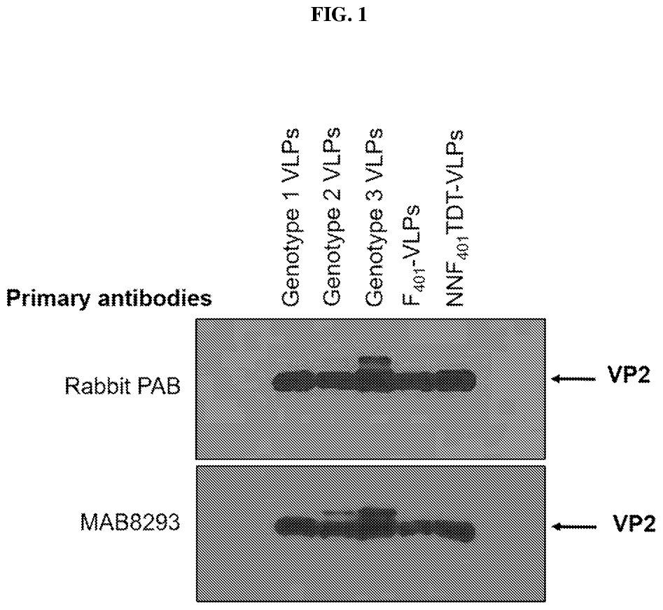

FIG. 2: Electron micrographs of purified VLPs of parvovirus B19. (A) Genotype 1, (B) Genotype 2 and (C) Genotype 3.

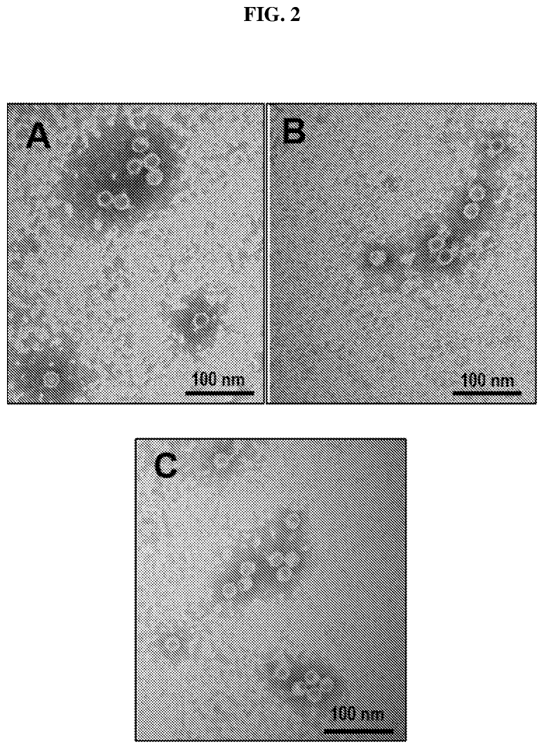

FIG. 3: The detection profile of B19V protein using different cloned hybridoma cell lines. These immunoblots represent the detection profile of different MAbs generated against mutated B19V N₃₉₉N₄₀₀F₄₀₁TDT₄₀₄-VLPs. Lanes 1-6 show six MAbs, which were reactive with both sequential and conformational epitopes of VLPs. Lanes 7, 10, and 11 show MAbs that were reactive only against conformational epitopes; they did not react with sequential epitopes of VP2 on the IB. The MAb #12 in lane 8 reacted partially to sequential epitopes of F₄₀₁-VLPs. The positive control (PC; lane 9) was the commercially available MAB8293. The arrow indicates the target VP2 protein band at 58 kDa.

FIG. 4: Hemagglutination inhibition assay (HIA) of MAbs. Three MAbs (#19B, #25 and #61) inhibited HA while the rest could not. Here, HA was indicated by a dark red pellet or button in the center of the well. The button appearance occurs because the red blood cells were not held in the agglutinated lattice structure and therefore settled into the low point of the U-bottom well. Rabbit polyclonal antibodies (PAB) raised against wtVLPs did not inhibit HA. PAB (rabbit polyclonal antibody); NC* (negative control) did not contain either VLPs or MAbs.

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include virus-like particle (VLP) binding agents, and related polynucleotides, cells, methods of making, and compositions. Other embodiments of the invention include methods of detecting VLPs, parvovirus, erythrovirus or parvovirus B19 using a VLP binding agent and diagnostic methods for parvovirus, erythrovirus or parvovirus B19. Further embodiments include methods for administering VLP binding agents to an animal. Other embodiments include treating parvovirus, erythrovirus or parvovirus B19 infections and other diseases. Additional embodiments of the invention are also discussed.

Parvovirus is the common name used to refer to all of the viruses in the Parvoviridae family Erythrovirus is a genus of the Parvoviridae family containing viruses that infect erythrocyte progenitor cells. Parvoviruses can infect many animals (e.g., mammals, porcine, canine, feline, primates, monkeys, and humans). Parvoviruses B19 belongs to erythroviruses, and contains three genotypes (Servant-Delmas et al., J Virol. (October 2010) Vol. 84, No. 19, pp. 9658-9665). Genotype 1 includes prototype B19 and two genotypes. Genotype 2 includes the Lali strain and the A6 strain, genotype 3a the V9 strain, and genotype 3b the D91.1 strain. In certain instances, the clinical spectrum associated with genotype 2 or 3 virus infection can be similar. Parvoviruses have VP1 and VP2 capsid proteins; VLPs can be formed either VP2 alone or together with VP1.

Parvovirus B19 consists of approximately 5.6 kb single-stranded genomic DNA (NCBI reference sequence NC_000883.2) that encodes one nonstructural protein (NS1), two structural proteins (VP1 and VP2), and 7.5 and 11 KD proteins. Genes spanning from nt 2624 to nt 4969 encode VP1 (minor) and VP2 (major) capsid proteins. VP2 protein (58 KD) overlaps C-terminus of VP1, and is composed of at least 95% of capsid. VP1 protein (81 KD) is 227 amino acids longer than VP2 and consists of only 5% of capsid proteins. P antigen is a cellular receptor of Parvovirus B19; Ku80 autoantigen and α5β1 intergrin are co-receptors for the entry of Parvovirus B19 into cells. In some instances, P antigen binding by parvovirus B19 can result in hemagglutination and thus can block access of B cells to parvovirus B19; this lack of access can, in some instances, prevent an immune response.

Some aspects of this disclosure are related to WO 2015/138424 A1, published Sep. 17, 2015, which is herein incorporated by reference in its entirety.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc.

In some embodiments, an antibody is a non-naturally occurring antibody. In some embodiments, an antibody is purified from natural components. In some embodiments, an antibody is recombinantly produced. In some embodiments, an antibody is produced by a hybridoma.

A “blocking” antibody or an “antagonist” antibody is one which inhibits or reduces biological activity of the antigen it binds, such as wtVLP, mVLP (e.g., as described in US 2017/0015712 A1, which is herein incorporated by reference in its entirety), a parvovirus, an erythrovirus, or a parvovirus B19. In a certain embodiment, blocking antibodies or antagonist antibodies substantially or completely inhibit the biological activity of the antigen. Desirably, the biological activity is reduced by 10%, 20%, 30%, 50%, 70%, 80%, 90%, 95%, or even 100%.

The term “anti-mVLP antibody” or “an antibody that binds to mVLP” refers to an antibody that is capable of binding an mVLP with sufficient affinity such that the antibody is useful as a diagnostic and/or therapeutic agent in targeting mVLP. Unless otherwise specified, the extent of binding of an anti-mVLP antibody to an unrelated, non-mVLP particle or unrelated protein is less than about 10% of the binding of the antibody to mVLP as measured, e.g., by a radioimmunoassay (RIA). In certain embodiments, an antibody that binds to mVLP has a dissociation constant (Kd) of ≤1 μM, ≤100 nM, ≤10 nM, ≤1 nM, or ≤0.1 nM. In other embodiments, the anti-mVLP antibody does not bind to other VLPs, such as VLPs made with VP1.

The term “anti-wtVLP antibody” or “an antibody that binds to wtVLP” refers to an antibody that is capable of binding an wtVLP with sufficient affinity such that the antibody is useful as a diagnostic and/or therapeutic agent in targeting wtVLP. Unless otherwise specified, the extent of binding of an anti-wtVLP antibody to an unrelated, non-wtVLP particle or unrelated protein is less than about 10% of the binding of the antibody to wtVLP as measured, e.g., by a radioimmunoassay (RIA). In certain embodiments, an antibody that binds to wtVLP has a dissociation constant (Kd) of ≤1 μM, ≤100 nM, ≤10 nM, ≤1 nM, or ≤0.1 nM. In other embodiments, the anti-wtVLP antibody does not bind to other VLPs, such as VLPs made with VP1.

The term “anti-parvovirus antibody”, “an antibody that binds to parvovirus”, “anti-erythrovirus antibody”, “an antibody that binds to erythrovirus”, “anti-parvovirus B19 antibody”, “an antibody that binds to parvovirus B19”, refers to an antibody that is capable of binding parvovirus, erythrovirus, or parvovirus B19, respectively, with sufficient affinity such that the antibody is useful as a diagnostic and/or therapeutic agent in targeting parvovirus, erythrovirus, or parvovirus B19. Unless otherwise specified, the extent of binding of an such an antibody to an unrelated particle or unrelated virus is less than about 10% of the binding of the antibody to parvovirus, erythrovirus, or parvovirus B19, respectively, as measured, e.g., by a radioimmunoassay (RIA). In certain embodiments, an antibody that binds to parvovirus, erythrovirus, or parvovirus B19 has a dissociation constant (Kd) of ≤1 μM, ≤100 nM, ≤10 nM, ≤1 nM, or ≤0.1 nM. In other embodiments, such antibodies do not bind to other VLPs, such as VLPs made with VP2.

The term “antibody fragment” is a subset of antigen binding fragments and refers to a portion of an intact antibody and refers to the antigenic determining variable regions of an intact antibody. Examples of antibody fragments include, but are not limited to, Fab, Fab′, F(ab′)₂, and Fv fragments, linear antibodies, single chain antibodies, and multispecific antibodies formed from antibody fragments. The term “antigen binding fragment” of an antibody includes one or more fragments of an antibody that retain the ability to specifically bind to an antigen. In some embodiments, the antigen-binding function of an antibody can be performed by certain fragments of a full-length antibody. Examples of binding fragments encompassed within the term “antigen binding fragment” of an antibody include (without limitation): (i) a Fab fragment, a monovalent fragment consisting of the VL, VH, CL and CH1 domains (e.g., an antibody digested by papain yields three fragments: two antigen-binding Fab fragments, and one Fc fragment that does not bind antigen); (ii) a F(ab′)₂ fragment, a bivalent fragment comprising two Fab fragments linked by a disulfide bridge at the hinge region (e.g., an antibody digested by pepsin yields two fragments: a bivalent antigen-binding F(ab′)₂ fragment, and a pFc′ fragment that does not bind antigen) and its related F(ab′) monovalent unit; (iii) an Fd fragment consisting of the VH and CH1 domains (i.e., that portion of the heavy chain which is included in the Fab); (iv) an Fv fragment consisting of the VL and VH domains of a single arm of an antibody, and the related disulfide linked Fv; (v) a dAb (domain antibody) or sdAb (single domain antibody) fragment (WARD et al. (1989) “Binding activities of a repertoire of single immunoglobulin variable domains secreted from Escherichia coli” Nature, Vol. 341, pp. 544-546, which is herein incorporated by reference in its entirety), which consists of a VH domain; and (vi) an isolated complementarity determining region (CDR).

A “monoclonal antibody” refers to a homogeneous antibody population involved in the highly specific recognition and binding of a single antigenic determinant, or epitope. This is in contrast to polyclonal antibodies that typically include different antibodies directed against different antigenic determinants. The term “monoclonal antibody” encompasses both intact and full-length monoclonal antibodies as well as antibody fragments (such as Fab, Fab′, F(ab′)₂, Fv), single chain (scFv) mutants, fusion proteins comprising an antibody portion, and any other modified immunoglobulin molecule comprising an antigen recognition site. Furthermore, “monoclonal antibody” refers to such antibodies made in any number of manners including but not limited to by hybridoma, phage selection, recombinant expression, and transgenic animals.

The term “humanized antibody” refers to forms of non-human (e.g., murine) antibodies that are specific immunoglobulin chains, chimeric immunoglobulins, or fragments thereof that contain minimal non-human (e.g., murine) sequences. In some embodiments, humanized antibodies are human immunoglobulins in which residues from the complementary determining region (CDR) are replaced by residues from the CDR of a non-human species (e.g., mouse, rat, rabbit, hamster) that have the desired specificity, affinity, and capability (JONES et al. (1986) “Replacing the complementarity-determining regions in a human antibody with those from a mouse” Nature, Vol. 321, pp. 522-525, which is herein incorporated by reference in its entirety; RIECHMANN et al. (1988) “Reshaping human antibodies for therapy” Nature, Vol. 332, pp. 323-327, which is herein incorporated by reference in its entirety; VERHOEYEN et al. (1988) “Reshaping human antibodies: grafting an antilysozyme activity” Science, Vol. 239, pp. 1534-1536, which is herein incorporated by reference in its entirety). In some instances, the Fv framework region (FR) residues of a human immunoglobulin are replaced with the corresponding residues in an antibody from a non-human species that has the desired specificity, affinity, and capability. The humanized antibody can sometimes be further modified by the substitution of additional residues either in the Fv framework region and/or within the replaced non-human residues to refine and optimize antibody specificity, affinity, and/or capability. In some embodiments, the humanized antibody will comprise substantially all of at least one, and typically two or three, variable domains containing all or substantially all of the CDR regions that correspond to the non-human immunoglobulin whereas all or substantially all of the FR regions are those of a human immunoglobulin consensus sequence. In other embodiments, the humanized antibody can also comprise at least a portion of an immunoglobulin constant region or domain (Fc), typically that of a human immunoglobulin. Some examples of methods used to generate humanized antibodies are described in U.S. Pat. No. 5,225,539 (which is herein incorporated by reference in its entirety), U.S. Pat. No. 5,639,641 (which is herein incorporated by reference in its entirety), ROGUSKA et al. (1994) “Humanization of murine monoclonal antibodies through variable domain resurfacing” Proc. Natl. Acad. Sci., USA, Vol. 91, No. 3, pp. 969-973 (which is herein incorporated by reference in its entirety), and ROGUSKA et al. (1996) “A comparison of two murine monoclonal antibodies humanized by CDR-grafting and variable domain resurfacing” Protein Eng., Vol. 9, No. 10, pp. 895-904 (which is herein incorporated by reference in its entirety). In some embodiments, a “humanized antibody” is a resurfaced antibody. In some embodiments, a “humanized antibody” is a CDR-grafted antibody.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., KABAT et al. Sequences of Proteins of Immunological Interest, (5th ed., 1991, National Institutes of Health, Bethesda Md.), which is herein incorporated by reference in its entirety); and (2) an approach based on crystallographic studies of antigen-antibody complexes (AL-LAZIKANI et al (1997) “Standard conformations for the canonical structures of immunoglobulins” J. Mol. Biol., Vol. 273, pp. 927-948), which is herein incorporated by reference in its entirety). In addition, combinations of these two approaches are sometimes used in the art to determine CDRs.

The Kabat numbering system is generally used when referring to a residue in the variable domain (approximately residues 1-107 of the light chain and residues 1-113 of the heavy chain) (e.g., KABAT et al., Sequences of Immunological Interest. 5th Ed. Public Health Service, National Institutes of Health, Bethesda, Md. (1991), which is herein incorporated by reference in its entirety).

The amino acid position numbering as in Kabat, refers to the numbering system used for heavy chain variable domains or light chain variable domains of the compilation of antibodies in KABAT et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FR or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues can be determined for a given antibody by alignment at regions of homology of the sequence of the antibody with a “standard” Kabat numbered sequence. Chothia refers instead to the location of the structural loops (CHOTHIA et al. (1987) “Canonical structures for the hypervariable regions of immunoglobulins” J. Mol. Biol., Vol. 196, pp. 901-917, which is herein incorporated by reference in its entirety). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The term “human antibody” means an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide such as, for example, an antibody comprising murine light chain and human heavy chain polypeptides.

The term “chimeric antibodies” refers to antibodies wherein the amino acid sequence of the immunoglobulin molecule is derived from two or more species. In certain embodiments, the variable region of both light and heavy chains corresponds to the variable region of antibodies derived from one species of mammals (e.g., mouse, rat, rabbit, etc.) with the desired specificity, affinity, and capability while the constant regions are homologous to the sequences in antibodies derived from another (usually human) to avoid eliciting an immune response in that species.

The terms “epitope” or “antigenic determinant” are used interchangeably herein and refer to that portion of an antigen capable of being recognized and specifically bound by a particular antibody. When the antigen is a polypeptide, epitopes can be formed, for example, both from contiguous amino acids and noncontiguous amino acids juxtaposed by folding (e.g., tertiary folding) of a protein. Epitopes formed from contiguous amino acids can sometimes be retained upon protein denaturing, whereas epitopes formed by folding (e.g., tertiary folding) can sometimes be lost upon protein denaturing. In some embodiments, an epitope can include at least 3, at least 5, or from 8 to 10 amino acids in a unique spatial conformation. In certain embodiments, the epitope is a conformational epitope (e.g., formed by folding (e.g., tertiary folding) of the polypeptide). In other embodiments, the epitope is not a conformational epitope.

“Binding affinity” generally refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd) or the half-maximal effective concentration (EC50). Affinity can be measured using any suitable method including, but not limited to those known in the art and those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present invention. Specific illustrative embodiments are described herein.

In some embodiments, an antibody or an antigen binding fragment thereof disclosed herein can specifically bind antigen (e.g., a parvovirus, an erythrovirus, or a parvovirus B19, wtVLP, mVLP, wild type VP2, or a polypeptide comprising a VP2 polypeptide with at least one amino acid modification relative to wild type VP2). In this context “specifically binds” means that the antibody (or an antigen binding fragment thereof) recognizes and binds to an antigen with greater affinity than to other, non-specific molecules that are not the antigen. For example, an antibody raised against or designed to target an antigen (such as a parvovirus, an erythrovirus, a parvovirus B19, wtVLP, mVLP, wild type VP2, or a polypeptide comprising a VP2 polypeptide with at least one amino acid modification relative to wild type VP2) or an antigen binding fragment thereof, to which it binds more efficiently than to a non-specific antigen can be described as specifically binding to the antigen. In some embodiments, an antibody or an antigen binding fragment thereof may have from about 2 to about 1000-fold (or from about 100 to about 1000-fold) specificity for the antigen compared to a non-antigen. In some embodiments, an antibody or an antigen binding fragment thereof can bind to an antigen with an Kd that is lower than 1×10⁻⁶ M, 1×10⁻⁷ M, 1×10⁻⁸M, 1×10⁻⁹ M, 1×10⁻¹⁰ M, 1×10⁻¹¹M, 1×10⁻¹²M, or less. Binding specificity can be determined using, for example, an enzyme-linked immunosorbant assay (ELISA), a radioimmunoassay (RIA), or a western blot assay using methodology well known in the art.

VP2 Polypeptides and Virus Like Particles (VLPs)

In some embodiments, VP2 polypeptides used in this disclosure include a VP2 polypeptide that has at least one amino acid modification relative to wild type VP2 (“wtVP2”). The term “VP2 polypeptide” encompasses mutant VP2 polypeptides (e.g., with one or more modifications made to a wtVP2 polypeptide) and wtVP2 polypeptides. A wtVP2 polypeptide can, in some embodiments, be a wild type VP2 polypeptide from a parvovirus, a wild type VP2 polypeptide from an erythrovirus, or a wild type VP2 polypeptide from a parvovirus B19. A wtVP2 polypeptide can, in some embodiments, be genotype 1 (e.g., SEQ ID NO:1), genotype 2 (e.g., Lali strain, SEQ ID NO:53), or genotype 3 (e.g., V9 strain, SEQ ID NO:54). One or more modifications, in some instances, can include an insertion, a deletion, a substitution, or combinations thereof.

In some embodiments, one or more modifications can occur at a wtVP2 (e.g., a B19 wtVP2) binding site or a virion (e.g., B19 virion) binding site, or a P antigen binding site. In some instances, one or more modifications to wtVP2 (e.g., SEQ ID NO:1) can include a substitution at Y401, a deletion at Y401, a substitution at Q399, a deletion at Q399, a substitution at Q400, a deletion at Q400, a substitution at Q404, a deletion at Q404, a substitution at Q368, a deletion at Q368, a substitution at Q369, a deletion at Q369, a substitution at Y392, a deletion at Y392, or combinations thereof. In yet other embodiments, one or more modifications to wtVP2 can include Y401F, Y401W, Y401A, Q368A, Q369A, Q368N, Q369N, Q399N, Q400N, Q404T, Y392A, Y392F, Q404N, Y401P, T402A, D403A, Q404A, or combinations thereof. In still other embodiments, the VP2 polypeptide is construct A, construct B, construct C, construct D, construct E, construct F, construct G, construct H, construct I, construct J, or construct K (see Table A). In some embodiments, the VP2 polypeptide does not include construct J. In certain embodiments, VP2 polypeptides used in this disclosure include: genotype 1 (e.g., SEQ ID NO:1), genotype 2 (e.g., Lali strain, SEQ ID NO:53), genotype 3 (e.g., V9 strain, SEQ ID NO:54), construct A (i.e., Y401F of SEQ ID NO:1), construct B (Y401W of SEQ ID NO:1), construct D (Q368A and Q369A of SEQ ID NO:1), and construct F (Q399N, Q400N, Y401F, and Q404T of SEQ ID NO:1).

TABLE A
Wild Type and mVP2

| Construct | | Amino acids |
|---|---|---|
| designation: | Mutation | (s) mutated |
| B19 Wild Type | none | none |
| (SEQ ID NO: 1) | | |
| A | QQYTDQ to QQFTDQ | Y401F |
| (SEQ ID NO: 42) | | |
| B | QQYTDQ to QQWTDQ | Y401W |
| (SEQ ID NO: 43) | | |
| C | QQYTDQ to QQATDQ | Y401A |
| (SEQ ID NO: 44) | | |
| D | KEYQQ to KEYAA | Q368A and Q369A |
| (SEQ ID NO: 45) | | |
| E | KEYQQ to KEYNN | Q368N and Q369N |
| (SEQ ID NO: 46) | | |
| F | QQYTDQ to NNFTDT | Q399N, Q400N, |
| (SEQ ID NO: 47) | | Y401F, and Q404T |
| G | QQYTDQ to NNYTDT | Q399N, Q400N, |
| (SEQ ID NO: 48) | | and Q404T |
| H | HTYFPN to HTAFPN | Y392A |
| (SEQ ID NO: 49) | | |
| I | HTYFPN to HTFFPN | Y392F |
| (SEQ ID NO: 50) | | |
| J | QQYTDQ to NNFTDN | Q399N, Q400N, |
| (SEQ ID NO: 51) | | Y401F and Q404N |
| K | QQYTDQ to NNYTDN | Q399N, Q400N, |
| (SEQ ID NO: 52) | | and Q404N |

In some embodiments of the mutant VP2 polypeptide, the wild type VP2 has the amino acid sequence of SEQ ID NO: 1, and the at least one amino acid modification comprises a substitution at Y401, a substitution at Q399, a substitution at Q400, a substitution at Q404, a substitution at Q368, a substitution at Q369, a substitution at Y392, Y401F, Y401W, Y401A, Q368A, Q369A, Q368N, Q369N, Q399N, Q400N, Q404T, Y392A, Y392F, Q404N, Y401P, T402A, D403A, Q404A, or combinations thereof. In other embodiments, the at least one amino acid modification comprises one or more of Y401F, Y401W, Q368A, Q369A, Q399N, Q400N, or Q404T. In certain embodiments, the VP2 polypeptide does not include construct J. In still other embodiments, the VP2 polypeptide is construct A, construct B, construct D, or construct F.

In certain embodiments, the VP2 polypeptides disclosed herein can be part of VLPs for any use disclosed herein, including but not limited to detection by VLP binding agent, methods of diagnosing, method of treating disease, and methods of inducing an immune response.

In some embodiments, the VP2 polypeptides disclosed herein are part of VLPs that can be detected by VLP binding agents or can be used in a diagnostic method. In certain embodiments, VP2 polypeptides that are part of VLPs that are detected by VLP binding agents or used in a diagnostic method include genotype 1 (e.g., SEQ ID NO:1), genotype 2 (e.g., Lali strain, SEQ ID NO:53), genotype 3 (e.g., V9 strain, SEQ ID NO:54), construct A (i.e., Y401F of SEQ ID NO:1), construct B (Y401W of SEQ ID NO:1), construct D (Q368A and Q369A of SEQ ID NO:1), and construct F (Q399N, Q400N, Y401F, and Q404T of SEQ ID NO:1)

In other embodiments, the VP2 polypeptides disclosed herein are part of VLPs that can be used in a variety of methods including, but not limited to methods of treating (e.g., any of the diseases disclosed herein) and methods of inducing an immune response. In certain embodiments, such VP2 polypeptides are mutant VP2 where the wild type VP2 has the amino acid sequence of SEQ ID NO: 1 and the at least one amino acid modification (1) comprises (a) Y401F and (b) Q399N or Q404T, (2) is Y401F, (3) is Q368A and Q369A, (4) is Q399N, Q400N, and Q404T, or (5) is Y392A. In some embodiments, the VP2 polypeptide is not construct J. In other embodiments, the VP2 polypeptide is selected from the group consisting of construct A, construct D, construct F, construct G, and construct H.

A VLP is a small particle that comprises one or more polypeptides from the outer coat (e.g., capsid) of a virus. VLPs do not contain any genetic material from the virus and thus cannot cause an infection. The expression of some viral structural proteins (e.g., envelope or capsid proteins) can result in the self-assembly of VLPs. As defined herein, unless otherwise indicated, “wtVLP” (also referred to as “wild type VLP”) is a VLP made only from VP2 proteins (i.e., with no modifications to the VP2 amino acid sequence); wtVLPs do not include any other proteins other than VP2 (i.e., VP1 is not included). As defined herein, unless otherwise indicated, “mVLP” (also referred to as “mutant VLP”) is a virus-like particle formed from inventive polypeptides, where the inventive polypeptide has at least one amino acid modification relative to wild type VP2. In certain embodiments, the mVLP can be morphologically similar to wtVLP (e.g., as determined using electron microscopy). In some embodiments, the mVLP can have reduced binding to P antigen compared to wtVLP (e.g., as measured using a hemagglutination assay). In some embodiments, the mVLP can have no detectable binding to P antigen (e.g., as measured using a hemagglutination assay). In other embodiments, the mVLP has reduced hemagglutination of red blood cells compared to wtVLP (e.g., as measured using a hemagglutination assay). In still other embodiments, the mVLP has no detectable hemagglutination (e.g., as measured using a hemagglutination assay). In certain embodiments, the mVLP can have one or more neutralizing epitopes (e.g., conformational epitopes or sequential epitopes) which can be determined by any suitable method (e.g., by using a hemagglutination inhibition assay or neutralization assay). In some embodiments, an epitope is a region on the surface of the mVLP (e.g., a conformational change in an mVLP can create or induce the appearance of an epitope) capable of eliciting an immune response; in certain embodiments, a neutralizing epitope is an epitope (e.g., a conformation epitope or sequential epitope) that can induce an immune response reactive to an mVLP, a wtVLP, a parvovirus, an erythrovirus, a B19 parvovirus, or combinations thereof; in some embodiments, the neutralizing epitope is an epitope (e.g., a conformation epitope or sequential epitope) that can neutralize the infectivity of a parvovirus, an erythrovirus, a B19 parvovirus, or combinations thereof. In some embodiments, the epitope is created one or more mVP2s of the mVLP (e.g., one or more VP2 on the surface of the mVLP) capable of eliciting an immune response; in certain embodiments, a neutralizing epitope is an epitope created by a conformation epitope that can induce an immune response reactive to an mVLP, a wtVLP, a parvovirus, an erythrovirus, a B19 parvovirus, or combinations thereof. In other embodiments, the mVLP can induce the production of antibodies (e.g., a high titer of antibodies) in an animal (e.g., mammals, humans, rats, mice, feline, canine, porcine, monkeys, or primates) where the antibodies are capable of inhibiting hemagglutination by wtVLP (e.g., as determined using a hemagglutination inhibition assay).

In certain embodiments, the VLPs can be made from any VP2 polypeptide disclosed herein and can be used for any use disclosed herein, including but not limited to detection by VLP binding agent, methods of diagnosing, method of treating disease, and methods of inducing an immune response.

In some embodiments, the VLPs can be detected by VLP binding agents or can be used in a diagnostic method. In certain embodiments, VP2 polypeptides that are part of VLPs that are detected by VLP binding agents or used in a diagnostic method include genotype 1 (e.g., SEQ ID NO:1), genotype 2 (e.g., Lali strain, SEQ ID NO:53), genotype 3 (e.g., V9 strain, SEQ ID NO:54), construct A (i.e., Y401F of SEQ ID NO:1), construct B (Y401W of SEQ ID NO:1), construct D (Q368A and Q369A of SEQ ID NO:1), and construct F (Q399N, Q400N, Y401F, and Q404T of SEQ ID NO:1)

In other embodiments, the VLPs can be used in a variety of methods including, but not limited to methods of treating (e.g., any of the diseases disclosed herein) and methods of inducing an immune response. In certain embodiments, such VLP can be made from VP2 polypeptides that are mutant VP2 where the wild type VP2 has the amino acid sequence of SEQ ID NO: 1 and the at least one amino acid modification (1) comprises (a) Y401F and (b) Q399N or Q404T, (2) is Y401F, (3) is Q368A and Q369A, (4) is Q399N, Q400N, and Q404T, or (5) is Y392A. In some embodiments, the VP2 polypeptide is not construct J. In other embodiments, the VP2 polypeptide is selected from the group consisting of construct A, construct D, construct F, construct G, and construct H.

VLP Binding Agents

VLP binding agents specifically bind to a VLP (e.g., a wtVLP or a mVLP) as disclosed herein, a parvovirus, an erythrovirus, or a parvovirus B19. In other embodiments, VLP binding agents can be antibodies (e.g., monoclonal antibodies), antigen binding fragments (e.g., antibody fragments), immunoconjugates, or polypeptides. In yet other embodiments, the VLP binding agent specifically binds to a conformation epitope, sequence epitope, or a surface sequence epitope of a VLP, a parvovirus, an erythrovirus, or a parvovirus B19. In certain embodiments, the VLP binding agent can bind to an epitope of a VLP made from a VP2 polypeptide including: genotype 1 (e.g., SEQ ID NO:1), genotype 2 (e.g., Lali strain, SEQ ID NO:53), genotype 3 (e.g., V9 strain, SEQ ID NO:54), construct A (i.e., Y401F of SEQ ID NO:1), construct B (Y401W of SEQ ID NO:1), construct D (Q368A and Q369A of SEQ ID NO:1), and construct F (Q399N, Q400N, Y401F, and Q404T of SEQ ID NO:1)—See Table AA.

In some embodiments, the VLP binding agent comprises the heavy and light chain CDR sequences of antibody 19 (antibody 19 is referred to as antibody 19B in the EXAMPLES section), antibody 25, antibody 61, or antibody 91, shown in Tables 1 and 2.

TABLE 1
Variable Heavy Chain CDR Amino Acid Sequences

| Antibody | VH CDR1 | VH CDR2 | VH CDR3 |
|---|---|---|---|
| 19 | SYTMY | YVNPSSGYTNYNQKFK | HGNYRYDGYYGMDC |
| | (SEQ ID | D (SEQ ID NO: 3) | (SEQ ID NO: 4) |
| | NO: 2) | | |
| 25 | NYWMH | RIDPYDSETHYNQKFK | YYYGSNYVWHFDV |
| | (SEQ ID | D (SEQ ID NO: 6) | (SEQ ID NO: 7) |
| | NO: 5) | | |
| 61 | SYTMH | YINPSSGYTNYNQKFK | PDGYYAWFAY |
| | (SEQ ID | D (SEQ ID NO: 9) | (SEQ ID NO: |
| | NO: 8) | | 10) |
| 91 | DYFMN | RINPYNGDSFYNQNFR | YRYDGYAMDY |
| | (SEQ ID | G (SEQ ID NO: | (SEQ ID NO: |
| | NO: 11) | 12) | 13) |

TABLE 2
Variable Light Chain CDR Amino Acid Sequences

| Antibody | VL CDR1 | VL CDR2 | VL CDR3 |
|---|---|---|---|
| 19 | KASEDIYNRLA | GATSLET | QQYWSTPYT |
| | (SEQ ID NO: 14) | (SEQ ID | (SEQ ID NO: 16) |
| | | NO: 15) | |
| 25 | KSSQSLLDSDGRTYLN | LVSKLDS | WQGTHLPYT |
| | (SEQ ID NO: 17) | (SEQ ID | (SEQ ID NO: 19) |
| | | NO: 18) | |
| 61 | KASEDIYNRLA | GATSLET | QQYWSTPT |
| | (SEQ ID NO: 20) | (SEQ ID | (SEQ ID NO: 22) |
| | | NO: 21) | |
| 91 | RSSQSLVHSDGNTYLH | KVSNRFS | SQSTHVPFT |
| | (SEQ ID NO: 23) | (SEQ ID | (SEQ ID NO: 25) |
| | | NO: 24) | |

In other embodiments, the VLP binding agents can be antibodies (e.g., monoclonal antibodies) or antigen binding fragments (e.g., antibody fragments) that specifically bind to VLPs, parvoviruses, erythroviruses, or parvovirus B19, where the VLP binding agents comprise the CDRs antibody 19 (SEQ ID NOS:2-4 and 14-16) (e.g., each with up to four (e.g., 0, 1, 2, 3, or 4) conservative amino acid substitutions), the CDRs antibody 25 (SEQ ID NOS:5-7 and 17-19) (e.g., each with up to four (e.g., 0, 1, 2, 3, or 4) conservative amino acid substitutions), the CDRs antibody 61 (SEQ ID NOS:8-10 and 20-22) (e.g., each with up to four (e.g., 0, 1, 2, 3, or 4) conservative amino acid substitutions), or the CDRs antibody 91 (SEQ ID NOS:11-13 and 23-25 (e.g., each with up to four (e.g., 0, 1, 2, 3, or 4) conservative amino acid substitutions).

In some embodiments, polypeptides can comprise one of the individual variable light chains or variable heavy chains described herein. In certain embodiments, antibodies and polypeptides can also comprise both a variable light chain and a variable heavy chain. The variable light chain and variable heavy chain sequences of antibodies 19, 25, 61, and 91 are provided in Tables 3 and 4 below.

TABLE 3
Variable Heavy Chain Amino Acid Sequences

| Antibody | VH Amino Acid Sequence |
|---|---|
| 19 | QVQLQQSGAELARPGASVKMSCKASGYTFTSYTMYWVKQRP |
| | GQGLEWIGYVNPSSGYTNYNQKFKDKATLTADKSSSTAYMQ |
| | LSSLTSEDSAVYYCARHGNYRYDGYYGMDCWGQGTSVTVSS |
| | (SEQ ID NO: 26) |
| 25 | QVQLQQPGAELVRPGASVKLSCKASGYTFTNYWMHWVKQRP |
| | EQGLEWIGRIDPYDSETHYNQKFKDKAILTVDTSSNTAYMQ |
| | LSSLTSEDSAVYYCTSYYYGSNYVWHFDVWGAGTTVTVSS |
| | (SEQ ID NO: 27) |
| 61 | QVQLQQSGAELARPGASVKMSCKASGYTFTSYTMHWVKQRP |
| | GQGLEWIGYINPSSGYTNYNQKFKDKATLTADKSSSTAYMQ |
| | LSSLTSEDSAVYYCARPDGYYAWFAYWGQGTLVTVSA |
| | (SEQ ID NO: 28) |
| 91 | EVQLQQSGPELVKPGASVKISCKASGYSFTDYFMNWVKQSH |
| | GKSLEWIGRINPYNGDSFYNQNFRGSVTLTVDKSSGTAHME |
| | LLSLTSEDSTVYYCGAYRYDGYAMDYWGQGTSVTVSS |
| | (SEQ ID NO: 29) |

TABLE 4
Variable Light Chain Amino Acid Sequences

| Antibody | VL Amino Acid Sequence |
|---|---|
| 19 | DIQMTQSSSSFSVSLGDRVTITCKASEDIYNRLAWYQQKPG |
| | NAPRLLISGATSLETGVPSRFSGSGSGKDYTLSITSLQTED |
| | VATYYCQQYWSTPYTFGGGTKLEIK |
| | (SEQ ID NO: 30) |
| 25 | DVVMTQTPLTLSVTIGQPASISCKSSQSLLDSDGRTYLNWL |
| | LQRPGQSPKRLIYLVSKLDSGVPDRFTGSGSGTDFTLKISR |
| | VEAEDLGIYYCWQGTHLPYTFGGGTKLEIK |
| | (SEQ ID NO: 31) |
| 61 | DIQMTQSSSSFSVSLGDRVTITCKASEDIYNRLAWYQQKPG |
| | NAPRLLISGATSLETGVPSRFSGSGSGKDYTLSITSLQTED |
| | VATYYCQQYWSTPTFGGGTKLEIK |
| | (SEQ ID NO: 32) |
| 91 | DVVMTQTPLSLPVSLGDQASISCRSSQSLVHSDGNTYLHWY |
| | LQKPGQSPKLLIYKVSNRFSGVPDRFSGSGSGTDFTLKISR |
| | VEAEDLGVYFCSQSTHVPFTFGSGTKLEIK |
| | (SEQ ID NO: 33) |

In some embodiments, polypeptides comprise: (a) a polypeptide having at least about 90% sequence identity to any of SEQ ID NOs:26-29; and/or (b) a polypeptide having at least about 90% sequence identity to any of SEQ ID NOs:30-33. In certain embodiments, the polypeptide comprises a polypeptide having at least about 95%, at least about 96%, at least about 97%, at least about 98%, or at least about 99% sequence identity to any of SEQ ID NOs:26-33. In certain embodiments, the polypeptide comprises (a) a polypeptide having at least about 95% sequence identity to any of SEQ ID NOs:26-29 and/or (b) a polypeptide having at least about 95% sequence identity to any of SEQ ID NOs:30-33. In certain embodiments, the polypeptide comprises (a) a polypeptide having the amino acid sequence of any of SEQ ID NOs:27-29; and/or (b) a polypeptide having the amino acid sequence of any of SEQ ID NOs:30-33. In certain embodiments, the polypeptide is an antibody and/or the polypeptide specifically binds VLPs, parvoviruses, erythroviruses, or parvovirus B19. In certain embodiments, the polypeptide is a murine, chimeric, or humanized antibody that specifically binds VLPs, parvoviruses, erythroviruses, or parvovirus B19. In certain embodiments, the polypeptide having a certain percentage of sequence identity to SEQ ID NOs:26-33 differs from SEQ ID NOs:26-33, respectively, by conservative amino acid substitutions only. The amino acid sequence identity (e.g., percent identity) can be determined by any suitable method, such as using BLAST, BLAST-2, ALIGN, ALIGN-2, or Megalign software. Unless otherwise indicated, the amino acid sequence identity (e.g., percent identity) is determined using BLAST-2.

In some embodiments, the antibody (e.g., monoclonal antibody) or antibody fragment comprises an amino acid sequence that has: (a) at least about 90% sequence identity to any of SEQ ID NOs:26-29; and/or (b) at least about 90% sequence identity to any of SEQ ID NOs:30-33. In certain embodiments, the antibody (e.g., monoclonal antibody) or antibody fragment comprises an amino acid sequence that has at least about 95%, at least about 96%, at least about 97%, at least about 98%, or at least about 99% sequence identity to any of SEQ ID NOs:26-33. In certain embodiments, the antibody (e.g., monoclonal antibody) or antibody fragment comprises an amino acid sequence that has (a) at least about 95% sequence identity to any of SEQ ID NOs:26-29 and/or (b) at least about 95% sequence identity to any of SEQ ID NOs:30-33. In certain embodiments, the antibody (e.g., monoclonal antibody) or antibody fragment comprises (a) the amino acid sequence of any of SEQ ID NOs:27-29; and/or (b) the amino acid sequence of any of SEQ ID NOs:30-33. In certain embodiments, the antibody (e.g., monoclonal antibody) or antibody fragment specifically binds to VLPs, parvoviruses, erythroviruses, or parvovirus B19. In certain embodiments, the polypeptide is a human, murine, chimeric, or humanized antibody that specifically binds VLPs. In certain embodiments, the antibody (e.g., monoclonal antibody) or antibody fragment having a certain percentage of sequence identity to SEQ ID NOs:26-33 differs from SEQ ID NOs:26-33, respectively, by conservative amino acid substitutions only.

In certain embodiments, the polypeptides comprise a variable light chain that is at least about 85%, at least about 90%, at least about 95%, or at least about 99%, or is identical to the variable light chain sequence of the antibody produced by a hybridoma described herein. In other embodiments, the polypeptides comprise a variable heavy chain that is at least about 85%, at least about 90%, at least about 95%, or at least about 99%, or is identical to the variable heavy chain sequence of the antibody produced by a hybridoma described herein. In still other embodiments, the antibodies (e.g., monoclonal antibodies) or antibody fragments thereof comprise a variable light chain that is at least about 85%, at least about 90%, at least about 95%, or at least about 99%, or is identical to the variable light chain sequence of the antibody produced by a hybridoma described herein. In yet other embodiments, the antibodies (e.g., monoclonal antibodies) or antibody fragments thereof comprise a variable heavy chain that is at least about 85%, at least about 90%, at least about 95%, or at least about 99%, or is identical to the variable heavy chain sequence of the antibody produced by the hybridoma described herein. In some embodiments, the antibody or antigen binding fragment thereof is produced by a hybridoma described herein.

In certain embodiments, the polypeptide can comprise a light chain that is at least about 85%, at least about 90%, at least about 95%, or at least about 99%, or is identical to the light chain sequence of the antibody produced by a hybridoma described herein. In other embodiments, polypeptide can comprise a heavy chain that is at least about 85%, at least about 90%, at least about 95%, or at least about 99%, or is identical to the heavy chain sequence of the antibody (e.g., monoclonal antibody) produced by a hybridoma described herein.

In certain embodiments, the antibody (e.g., monoclonal antibody) or antigen binding fragments thereof can comprise heavy and light chain sequences that are at least about 85%, at least about 90%, at least about 95%, or at least about 99%, or identical to the heavy and light chain sequences of the antibody (e.g., monoclonal antibody) produced by a hybridoma described herein.

The affinity or avidity of an antibody for an antigen can be determined experimentally using any suitable method well known in the art, e.g., flow cytometry, enzyme-linked immunoabsorbent assay (ELISA), or radioimmunoassay (RIA), or kinetics (e.g., BIACORE™ analysis). Direct binding assays as well as competitive binding assay formats can be readily employed. (See, for example, Berzofsky, et al., “Antibody-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein. The measured affinity of a particular antibody-antigen interaction can vary if measured under different conditions (e.g., salt concentration, pH, temperature). Thus, measurements of affinity and other antigen-binding parameters (e.g., KD or Kd, Kₒₙ, K_{off}) are made with standardized solutions of antibody and antigen, and a standardized buffer, as known in the art and such as the buffer described herein.

Monoclonal antibodies can be prepared using hybridoma methods, such as those described by Kohler and Milstein (1975) Nature 256:495. Using the hybridoma method, a mouse, hamster, or other appropriate host animal, is immunized to elicit the production by lymphocytes of antibodies that will specifically bind to an immunizing antigen. Lymphocytes can also be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay (e.g., radioimmunoassay (RIA); enzyme-linked immunosorbent assay (ELISA)) can then be propagated either in vitro culture using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies.

Alternatively monoclonal antibodies can also be made using recombinant DNA methods as described in U.S. Pat. No. 4,816,567. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional procedures. The isolated polynucleotides encoding the heavy and light chains are then cloned into suitable expression vectors, which when transfected into host cells such as E. coli cells, simian COS cells, Chinese hamster ovary (CHO) cells, or myeloma cells that do not otherwise produce immunoglobulin protein, monoclonal antibodies are generated by the host cells. Also, recombinant monoclonal antibodies or fragments thereof of the desired species can be isolated from phage display libraries expressing CDRs of the desired species as described (McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the monoclonal antibody against the human VLP, parvovirus, erythrovirus, or parvovirus B19, is a humanized antibody. In certain embodiments, such antibodies are used therapeutically to reduce antigenicity and HAMA (human anti-mouse antibody) responses when administered to a human subject.

Methods for engineering, humanizing or resurfacing non-human or human antibodies can also be used and are well known in the art. A humanized, resurfaced or similarly engineered antibody can have one or more amino acid residues from a source that is non-human, e.g., but not limited to, mouse, rat, rabbit, non-human primate or other mammal. These non-human amino acid residues are replaced by residues that are often referred to as “import” residues, which are typically taken from an “import” variable, constant or other domain of a known human sequence.

Such imported sequences can be used to reduce immunogenicity or reduce, enhance or modify binding, affinity, on-rate, off-rate, avidity, specificity, half-life, or any other suitable characteristic, as known in the art. In some embodiments, the CDR residues are directly and most substantially involved in influencing VLP, parvovirus, erythrovirus, or parvovirus B19 binding. Accordingly, part or all of the non-human or human CDR sequences are maintained while the non-human sequences of the variable and constant regions can be replaced with human or other amino acids.

Antibodies can also optionally be humanized, resurfaced, engineered or human antibodies engineered with retention of high affinity for a VLP, a parvovirus, an erythrovirus, or a parvovirus B19, and other favorable biological properties. To achieve this goal, humanized (or human) or engineered antibodies (e.g., against a VLP, a parvovirus, an erythrovirus, or a parvovirus B19) and resurfaced antibodies (e.g., against a VLP, a parvovirus, an erythrovirus, or a parvovirus B19) can be optionally prepared by a process of analysis of the parental sequences and various conceptual humanized and engineered products using three-dimensional models of the parental, engineered, and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen, such as VLP. In this way, framework (FR) residues can be selected and combined from the consensus and import sequences so that the desired antibody characteristic, such as increased affinity for the target antigen(s), is achieved.

Humanization, resurfacing or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,639,641, 5,723,323; 5,976,862; 5,824,514; 5,817,483; 5,814,476; 5,763,192; 5,723,323; 5,766,886; 5,714,352; 6,204,023; 6,180,370; 5,693,762; 5,530,101; 5,585,089; 5,225,539; 4,816,567; PCT/: US98/16280; US96/18978; US91/09630; US91/05939; US94/01234; GB89/01334; GB91/01134; GB92/01755; WO90/14443; WO90/14424; WO90/14430; EP 229246; 7,557,189; 7,538,195; and U.S. Pat. No. 7,342,110, each of which is entirely incorporated herein by reference, including the references cited therein.

In certain alternative embodiments, the antibody to a VLP, a parvovirus, an erythrovirus, or a parvovirus B19, is a human antibody. Human antibodies can be directly prepared using various techniques known in the art. Immortalized human B lymphocytes immunized in vitro or isolated from an immunized individual that produce an antibody directed against a target antigen can be generated (See, e.g., Cole et al., Monoclonal Antibodies and Cancer Therapy, Alan R. Liss, p. 77 (1985); Boemer et al., 1991, J. Immunol., 147 (1):86-95; and U.S. Pat. No. 5,750,373). Also, the human antibody can be selected from a phage library, where that phage library expresses human antibodies, as described, for example, in Vaughan et al., 1996, Nat. Biotech., 14:309-314, Sheets et al., 1998, Proc. Nat'l. Acad. Sci., 95:6157-6162, Hoogenboom and Winter, 1991, J. Mol. Biol., 227:381, and Marks et al., 1991, J. Mol. Biol., 222:581). Techniques for the generation and use of antibody phage libraries are also described in U.S. Pat. Nos. 5,969,108, 6,172,197, 5,885,793, 6,521,404; 6,544,731; 6,555,313; 6,582,915; 6,593,081; 6,300,064; 6,653,068; 6,706,484; and 7,264,963; and Rothe et al., 2007, J. Mol. Bio., doi:10.1016/j.jmb.2007.12.018 (each of which is incorporated by reference in its entirety). Affinity maturation strategies and chain shuffling strategies (Marks et al., 1992, Bio/Technology 10:779-783, incorporated by reference in its entirety) are known in the art and can be employed to generate high affinity human antibodies.

Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In certain embodiments are provided an antibody fragment. Various techniques are known for the production of antibody fragments. Traditionally, these fragments are derived via proteolytic digestion of intact antibodies (for example Morimoto et al., 1993, Journal of Biochemical and Biophysical Methods 24:107-117; Brennan et al., 1985, Science, 229:81). In certain embodiments, antibody fragments are produced recombinantly. Fab, Fv, and scFv antibody fragments can all be expressed in and secreted from E. coli or other host cells, thus allowing the production of large amounts of these fragments. Such antibody fragments can also be isolated from antibody phage libraries. The antibody fragment can also be linear antibodies as described in U.S. Pat. No. 5,641,870, for example, and can be monospecific or bispecific. Other techniques for the production of antibody fragments will be apparent to the skilled practitioner.

For the purposes of the present invention, it should be appreciated that modified antibodies can comprise any type of variable region that provides for the association of the antibody with the polypeptides of a VLP, a parvovirus, an erythrovirus, or a parvovirus B19. In this regard, the variable region can comprise or be derived from any type of mammal that can be induced to mount a humoral response and generate immunoglobulins against the desired antigen. As such, the variable region of the modified antibodies can be, for example, of human, murine, non-human primate (e.g., cynomolgus monkeys, macaques, etc.) or lupine origin. In some embodiments both the variable and constant regions of the modified immunoglobulins are human. In other embodiments the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence changing. Although the CDRs can be derived from an antibody of the same class or even subclass as the antibody from which the framework regions are derived, it is envisaged that the CDRs will be derived from an antibody of different class and in certain embodiments from an antibody from a different species. It may not be necessary to replace all of the CDRs with the complete CDRs from the donor variable region to transfer the antigen-binding capacity of one variable domain to another. Rather, it may only be necessary to transfer those residues that are necessary to maintain the activity of the antigen-binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as reduced serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. That is, the modified antibodies disclosed herein can comprise alterations or modifications to one or more of the three heavy chain constant domains (CH1, CH2 or CH3) and/or to the light chain constant domain (CL). In some embodiments, modified constant regions wherein one or more domains are partially or entirely deleted are contemplated. In some embodiments, the modified antibodies will comprise domain deleted constructs or variants wherein the entire CH2 domain has been removed (ΔCH2 constructs). In some embodiments, the omitted constant region domain will be replaced by a short amino acid spacer (e.g., 10 residues) that provides some of the molecular flexibility typically imparted by the absent constant region.

It will be noted that in certain embodiments, the modified antibodies can be engineered to fuse the CH3 domain directly to the hinge region of the respective modified antibodies. In other constructs it may be desirable to provide a peptide spacer between the hinge region and the modified CH2 and/or CH3 domains. For example, compatible constructs could be expressed wherein the CH2 domain has been deleted and the remaining CH3 domain (modified or unmodified) is joined to the hinge region with a 5-20 amino acid spacer. Such a spacer can be added, for instance, to ensure that the regulatory elements of the constant domain remain free and accessible or that the hinge region remains flexible. However, it should be noted that amino acid spacers can, in some cases, prove to be immunogenic and elicit an unwanted immune response against the construct. Accordingly, in certain embodiments, any spacer added to the construct will be relatively non-immunogenic, or even omitted altogether, so as to maintain the desired biochemical qualities of the modified antibodies.

Besides the deletion of whole constant region domains, it will be appreciated that the antibodies of the present invention can be provided by the partial deletion or substitution of a few or even a single amino acid. For example, the mutation of a single amino acid in selected areas of the CH2 domain may be enough to substantially reduce Fc binding. Similarly, it may be desirable to simply delete that part of one or more constant region domains that control the effector function (e.g., complement C1Q binding) to be modulated. Such partial deletions of the constant regions can improve selected characteristics of the antibody (serum half-life) while leaving other desirable functions associated with the subject constant region domain intact. Moreover, as alluded to above, the constant regions of the disclosed antibodies can be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. Certain embodiments can comprise the addition of one or more amino acids to the constant region to enhance desirable characteristics such as decreasing or increasing effector function or provide for more cytotoxin or carbohydrate attachment. In such embodiments it can be desirable to insert or replicate specific sequences derived from selected constant region domains.

The present invention further embraces variants and equivalents which are substantially homologous to the chimeric, humanized and human antibodies, or antibody fragments thereof, set forth herein. These can contain, for example, conservative substitution mutations, i.e., the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art. For example, conservatively substituted sequence indicates that a given amino acid residue is replaced by a residue having similar physiochemical characteristics. Examples of conservative substitutions include substitution of one aliphatic residue for another, such as Ile, Val, Leu, or Ala for one another, or substitutions of one polar residue for another, such as between Lys and Arg; Glu and Asp; or Gln and Asn. Other such conservative substitutions include, for example, substitutions of entire regions having similar hydrophobicity characteristics.

In some embodiments, the shorter the length of the molecule, the fewer the changes that can be made within the molecule while retaining function. Longer domains may have an intermediate number of changes. The full-length protein will have the most tolerance for a larger number of changes. However, it must be appreciated that certain molecules or domains that are highly dependent upon their structure may tolerate little or no modification.

The polypeptides of the present invention can be recombinant polypeptides, natural polypeptides, or synthetic polypeptides comprising an antibody, or fragment thereof, against a VLP, a parvovirus, an erythrovirus, or a parvovirus B19. It will be recognized in the art that some amino acid sequences of the invention can be varied without significant effect of the structure or function of the protein. Thus, the invention further includes variations of the polypeptides which show substantial activity or which include regions of an antibody, or fragment thereof, against a human folate receptor protein. Such mutants include deletions, insertions, inversions, repeats, and type substitutions.

The polypeptides and analogs can be further modified to contain additional chemical moieties not normally part of the protein. Those derivatized moieties can improve the solubility, the biological half life or absorption of the protein. The moieties can also reduce or eliminate any desirable side effects of the proteins and the like. An overview for those moieties can be found in REMINGTON'S PHARMACEUTICAL SCIENCES, 20th ed., Mack Publishing Co., Easton, Pa. (2000).

The isolated polypeptides described herein can be produced by any suitable method known in the art. Such methods range from direct protein synthetic methods to constructing a DNA sequence encoding isolated polypeptide sequences and expressing those sequences in a suitable transformed host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g., Zoeller et al., Proc. Nat'l. Acad. Sci. USA 81:5662-5066 (1984) and U.S. Pat. No. 4,588,585.

In some embodiments a DNA sequence encoding a polypeptide of interest would be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest will be produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular isolated polypeptide of interest will be inserted into an expression vector and operatively linked to an expression control sequence appropriate for expression of the protein in a desired host. Proper assembly can be confirmed by nucleotide sequencing, restriction mapping, and expression of a biologically active polypeptide in a suitable host. As is well known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies, or fragments thereof, against VLP, parvovirus, erythrovirus, or parvovirus B19. Recombinant expression vectors are replicable DNA constructs which have synthetic or cDNA-derived DNA fragments encoding a polypeptide chain of an anti-VLP antibody, or fragment thereof, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

The choice of expression control sequence and expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages.

Suitable host cells for expression of a VLP-binding polypeptide or antibody (or a VLP, a parvovirus, an erythrovirus, or a parvovirus B19 to use as an antigen) include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are described by Pouwels et al. (Cloning Vectors: A Laboratory Manual, Elsevier, N.Y., 1985), the relevant disclosure of which is hereby incorporated by reference. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 04009823, each of which is hereby incorporated by reference herein in its entirety.

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include HEK-293 and HEK-293T, the COS-7 lines of monkey kidney cells, described by Gluzman (Cell 23:175, 1981), and other cell lines including, for example, L cells, C127, 3T3, Chinese hamster ovary (CHO), HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column Isolated proteins can also be physically characterized using such techniques as proteolysis, nuclear magnetic resonance and x-ray crystallography.

For example, supernatants from systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. Alternatively, an anion exchange resin can be employed, for example, a matrix or substrate having pendant diethylaminoethyl (DEAE) groups. The matrices can be acrylamide, agarose, dextran, cellulose or other types commonly employed in protein purification. Alternatively, a cation exchange step can be employed. Suitable cation exchangers include various insoluble matrices comprising sulfopropyl or carboxymethyl groups. Finally, one or more reversed-phase high performance liquid chromatography (RP-HPLC) steps employing hydrophobic RP-HPLC media, e.g., silica gel having pendant methyl or other aliphatic groups, can be employed to further purify a VLP-binding agent. Some or all of the foregoing purification steps, in various combinations, can also be employed to provide a homogeneous recombinant protein.

Recombinant protein produced in bacterial culture can be isolated, for example, by initial extraction from cell pellets, followed by one or more concentration, salting-out, aqueous ion exchange or size exclusion chromatography steps. High performance liquid chromatography (HPLC) can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005, each of which is hereby incorporated by reference herein in its entirety.

Polynucleotides

In certain embodiments, the invention encompasses polynucleotides comprising one or more polynucleotides that encode a polypeptide that specifically binds VLP. For example, the invention provides a polynucleotide comprising a nucleic acid sequence that encodes an antibody (e.g., to a VLP, a parvovirus, an erythrovirus, or a parvovirus B19) or encodes a fragment of such an antibody. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand. In some embodiments, the polynucleotide is a cDNA or a DNA lacking one more endogenous introns.

In some embodiments, a polynucleotide is a non-naturally occurring polynucleotide. In some embodiments, a polynucleotide is recombinantly produced.

In certain embodiments, the polynucleotides are isolated. In certain embodiments, the polynucleotides are substantially pure. In some embodiments, a polynucleotide is purified from natural components.

The invention provides a polynucleotide comprising a polynucleotide encoding a polypeptide comprising a sequence selected from the group consisting of SEQ ID NOs:2-33. Also provided is a polynucleotide encoding a polypeptide having at least about 95%, at least about 96%, at least about 97%, at least about 98%, or at least about 99% sequence identity to SEQ ID NOs:2-33. The polynucleotide sequence identity (e.g., percent identity) can be determined by any suitable method, such as using BLAST, BLAST-2, ALIGN, ALIGN-2, or Megalign software. Unless otherwise indicated, the polynucleotide sequence identity (e.g., percent identity) is determined using BLAST-2.

The invention further provides a polynucleotide comprising a sequence selected from those shown in Tables 5 and 6 below.

TABLE 5
Variable Heavy Chain Polynucleotide Sequences

| Antibody | VH Polynucleotide Sequence |
|---|---|
| 19 | CAGGTCCAGCTGCAGCAGTCTGGGGCTGAACTGGCAAGACC |
| | TGGGGCCTCAGTGAAGATGTCCTGCAAGGCTTCTGGCTACA |
| | CCTTTACTAGCTACACGATGTACTGGGTAAAACAGAGGCCT |
| | GGACAGGGTCTGGAGTGGATTGGATACGTTAATCCTAGTAG |
| | TGGTTATACTAATTACAATCAGAAGTTCAAGGACAAGGCCA |
| | CATTGACTGCAGACAAATCCTCCAGCACAGCCTACATGCAA |
| | CTGAGCAGCCTGACATCTGAGGACTCTGCAGTCTATTACTG |
| | TGCAAGACATGGGAACTATAGGTACGACGGGTACTATGGTA |
| | TGGACTGCTGGGGTCAAGGAACCTCAGTCACCGTCTCCTCA |
| | (SEQ ID NO: 34) |
| 25 | CAGGTCCAACTGCAGCAGCCTGGGGCTGAGCTGGTGAGGCC |
| | TGGGGCTTCAGTGAAGCTGTCCTGCAAGGCTTCTGGCTACA |
| | CGTTCACCAACTACTGGATGCACTGGGTTAAACAGAGGCCT |
| | GAGCAAGGCCTTGAGTGGATTGGAAGGATTGATCCTTACGA |
| | TAGTGAAACTCACTACAATCAAAAGTTCAAGGATAAGGCCA |
| | TATTGACTGTGGACACATCCCCAACACAGCCTACATGCAAC |
| | TCAGCAGCCTGACATCTGAGGACTCTGCGGTCTATTACTGT |
| | ACATCGTATTACTACGGTAGTAATTACGTTTGGCACTTCGA |
| | TGTCTGGGGCGCAGGGACCACGGTCACCGTCTCCTCA |
| | (SEQ ID NO: 35) |
| 61 | CAGGTCCAGCTGCAGCAGTCTGGGGCTGAACTGGCAAGACC |
| | TGGGGCCTCAGTGAAGATGTCCTGCAAGGCTTCTGGCTACA |
| | CCTTTACTAGCTACACGATGCACTGGGTAAAACAGAGGCCT |
| | GGACAGGGTCTGGAATGGATTGGATACATTAATCCTAGCAG |
| | TGGTTATACTAATTACAATCAGAAGTTCAAGGACAAGGCCA |
| | CATTGACTGCAGACAAATCCTCCAGCACAGCCTACATGCAA |
| | CTGAGCAGCCTGACATCTGAGGACTCTGCAGTCTATTACTG |
| | TGCAAGACCAGATGGTTACTACGCCTGGTTTGCTTACTGGG |
| | GCCAAGGGACTCTGGTCACTGTCTCTGCA |
| | (SEQ ID NO: 36) |
| 91 | GAGGTTCAGCTGCAGCAGTCTGGACCTGAGCTGGTGAAGCC |
| | TGGGGCTTCAGTGAAGATTTCCTGCAAGGCTTCTGGTTACT |
| | CATTTACTGACTACTTTATGAACTGGGTGAAGCAGAGCCAT |
| | GGAAAGAGCCTTGAGTGGATTGGACGTATTAATCCTTACAA |
| | TGGTGATAGTTTCTACAACCAGAACTTCAGGGGCAGTGTCA |
| | CATTGACTGTTGACAAATCCTCTGGCACAGCCCACATGGAG |
| | CTCCTGAGCCTGACATCTGAGGACTCTACAGTCTATTATTG |
| | TGGAGCCTATAGGTACGACGGGTATGCTATGGACTACTGGG |
| | GTCAAGGAACCTCAGTCACCGTCTCCTCA |
| | (SEQ ID NO: 37) |

TABLE 6
Variable Light Chain Polynucleotide Sequences

| Antibody | VL Polynucleotide Sequence |
|---|---|
| 19 | GACATCCAGATGACACAATCTTCATCCTCCTTTTCTGTATC |
| | TCTAGGAGACAGAGTCACCATTACTTGCAAGGCAAGTGAGG |
| | ACATATATAATCGGTTAGCCTGGTATCAGCAGAAACCAGGA |
| | AATGCTCCTAGGCTCTTAATATCTGGTGCAACCAGTTTGGA |
| | AACTGGGGTTCCTTCAAGATTCAGTGGCAGTGGATCTGGAA |
| | AGGATTACACTCTCAGCATTACCAGTCTTCAGACTGAAGAT |
| | GTTGCTACTTATTACTGTCAACAGTATTGGAGTACTCCGTA |
| | CACGTTCGGAGGGGGGACCAAGCTGGAAATAAAA |
| | (SEQ ID NO: 38) |
| 25 | GATGTTGTGATGACCCAGACTCCTCTCACTTTGTCGGTTAC |
| | CATTGGACAACCAGCCTCCATCTCTTGTAAGTCAAGTCAGA |
| | GCCTCTTAGATAGTGATGGAAGGACATATTTGAATTGGTTG |
| | TTACAGAGGCCAGGCCAGTCTCCAAAGCGCCTAATCTATCT |
| | GGTGTCTAAACTGGACTCTGGAGTCCCTGACAGGTTCACTG |
| | GCAGTGGATCAGGGACAGATTTCACACTGAAAATCAGCAGA |
| | GTGGAGGCTGAGGATTTGGGAATTTATTATTGCTGGCAAGG |
| | TACACATCTTCCTTACACGTTCGGAGGGGGGACCAAGCTGG |
| | AAATAAAA |
| | (SEQ ID NO: 39) |
| 61 | GACATCCAGATGACACAATCTTCATCCTCCTTTTCTGTATC |
| | TCTAGGAGACAGAGTCACCATTACTTGCAAGGCAAGTGAGG |
| | ACATATATAATCGGTTAGCCTGGTATCAGCAGAAACCAGGA |
| | AATGCTCCTAGGCTCTTAATATCTGGTGCAACCAGTTTGGA |
| | AACTGGGGTTCCTTCAAGATTCAGTGGCAGTGGATCTGGAA |
| | AGGATTACACTCTCAGCATTACCAGTCTTCAGACTGAAGAT |
| | GTTGCTACTTATTACTGTCAACAGTATTGGAGTACTCCGAC |
| | GTTCGGTGGAGGCACCAAGCTGGAAATCAAA |
| | (SEQ ID NO: 40) |
| 91 | GATGTTGTGATGACCCAAACTCCACTCTCCCTGCCTGTCAG |
| | TCTTGGAGATCAAGCCTCCATCTCTTGCAGATCTAGTCAGA |
| | GCCTTGTACACAGTGATGGAAACACCTATTTACATTGGTAC |
| | CTGCAGAAGCCAGGCCAGTCTCCAAAGCTCCTGATCTACAA |
| | AGTTTCCAACCGATTTTCTGGGGTCCCAGACAGGTTCAGTG |
| | GCAGTGGATCAGGGACAGATTTCACACTCAAGATCAGCAGA |
| | GTGGAGGCTGAGGATCTGGGAGTTTATTTCTGCTCTCAAAG |
| | TACACATGTTCCATTCACGTTCGGCTCGGGGACAAAGTTGG |
| | AAATAAAA |
| | (SEQ ID NO: 41) |

In certain embodiments, the polynucleotide can have at least about 95%, at least about 96%, at least about 97%, at least about 98%, or at least about 99% sequence identity to any one of SEQ ID NOs:34-41.

In some embodiments, the polynucleotide encodes a variable light chain that is at least about 85%, at least about 90%, at least about 95%, or at least about 99%, or is identical to the variable light chain sequence of the antibody produced by a hybridoma disclosed herein.

In certain embodiments, the polynucleotide comprises a variable light chain-encoding sequence that is at least about 85%, at least about 90%, at least about 95%, or at least about 99%, or is identical to the variable light chain-encoding sequence that encodes the variable light chain of the antibody produced by a hybridoma disclosed herein.

In some embodiments, the polynucleotide encodes a variable heavy chain that is at least about 85%, at least about 90%, at least about 95%, or at least about 99%, or is identical to the variable heavy chain sequence of the antibody produced by a hybridoma disclosed herein.

In certain embodiments, the polynucleotide comprises a variable heavy chain-encoding sequence that is at least about 85%, at least about 90%, at least about 95%, or at least about 99%, or is identical to the variable heavy chain-encoding sequence that encodes the variable heavy chain of the antibody produced by a hybridoma disclosed herein.

In certain embodiments the polynucleotides comprise the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to form the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments the polynucleotides comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used.

In other embodiments, the present invention further relates to variants of the hereinabove described polynucleotides encoding, for example, fragments, analogs, and derivatives.

In yet other embodiments, the polynucleotide variants can contain alterations in the coding regions, non-coding regions, or both. In some embodiments the polynucleotide variants contain alterations which produce silent substitutions, additions, or deletions, but do not alter the properties or activities of the encoded polypeptide. In some embodiments, nucleotide variants are produced by silent substitutions due to the degeneracy of the genetic code. Polynucleotide variants can be produced for a variety of reasons, e.g., to optimize codon expression for a particular host (change codons in the human mRNA to those preferred by a bacterial host such as E. coli).

In still other embodiments, vectors and cells comprising the polynucleotides described herein are also provided.

Detection Conjugates

In certain embodiments, the invention provides VLP binding agents (e.g., antibodies, monoclonal antibodies, antigen binding fragments, or antibody fragments) against VLPs, parvoviruses, erythroviruses, or parvoviruses B19, that are linked to at least one agent to form a detection conjugate (e.g., an antibody conjugate). In order to increase the efficacy of VLP binding agents (e.g., antibodies, monoclonal antibodies, antigen binding fragments, or antibody fragments) as diagnostic it is conventional to link or covalently bind or complex at least one desired molecule or moiety. Such a molecule or moiety may be, but is not limited to, at least one reporter molecule. A reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles and/or ligands, such as biotin.

Certain examples of antibody conjugates are those conjugates in which the antibody or antigen binding fragment thereof provided herein is linked to a detectable label. “Detectable labels” are compounds and/or elements that can be detected due to their specific functional properties, and/or chemical characteristics, the use of which allows the antibody or antigen binding fragment to which they are attached to be detected, and/or further quantified if desired.

Many appropriate imaging agents are known in the art, as are methods for their attachment to antibodies (see, e.g., U.S. Pat. Nos. 5,021,236; 4,938,948; and 4,472,509, each incorporated herein by reference). The imaging moieties used can be paramagnetic ions; radioactive isotopes; fluorochromes; NMR-detectable substances; and/or X-ray imaging, for example.

Exemplary fluorescent labels contemplated for use as binding agent (e.g., antibody) conjugates include Alexa 350, Alexa 430, Alexa 488, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Dylight 488, Fluorescein Isothiocyanate (FITC), Green fluorescent protein (GFP), HEX, 6-JOE, Oregon Green 488, Oregon Green 500, Oregon Green 514, Pacific Blue, Phycoerythrin, REG, Rhodamine Green, Rhodamine Red, tetramethyl rhodamin (TMR) Renographin, ROX, TAMRA, TET, Tetramethylrhodamine, Texas Red, and derivatives of these labels (i.e., halogenated analogues, modified with isothiocynate or other linker for conjugating, etc.), for example. An exemplary radiolabel is tritium.

Antibody or antigen binding fragment detection conjugates contemplated in the present invention include those for use in vitro, where the antibody or fragment is linked to a secondary binding ligand and/or to an enzyme (an enzyme tag) that will generate a colored product upon contact with a chromogenic substrate. The VLP antibodies and antigen binding fragments thereof provided herein are particularly useful for conjugates methods because, for example, they are able to detect a dynamic range of VLPs, parvoviruses, erythroviruses, or a parvoviruses B19. Examples of suitable enzymes include urease, alkaline phosphatase, (horseradish) hydrogen peroxidase and/or glucose oxidase. In some embodiments, secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Molecules containing azido groups may also be used to form covalent bonds to proteins through reactive nitrene intermediates that are generated by low intensity ultraviolet light (Potter & Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; and Dholakia et al., 1989) and can be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the binding agent (e.g., antibody) (U.S. Pat. Nos. 4,472,509 and 4,938,948, each incorporated herein by reference). Monoclonal antibodies may also be reacted with an enzyme in the presence of a coupling agent such as glutaraldehyde or periodate. Protein binding (e.g., antibody) conjugates with fluorescein markers are prepared in the presence of these coupling agents or by reaction with an isothiocyanate. In U.S. Pat. No. 4,938,948, imaging of breast tumors, for example, is achieved using monoclonal antibodies, and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region, have also been disclosed in the literature (O'Shannessy et al., 1987).

In other embodiments of the invention, immunoglobulins are radiolabeled with nuclides such as tritium. In additional embodiments, nanogold particles (such as sizes from about 0.5 nm-40 nm) and/or Quantum Dots (Hayward, Calif.) are employed.

When a sandwich assay format is used, the capture antibody will be unlabeled. The detection antibody will be either directly labeled, or detected indirectly by addition (after washing off excess detection antibody) of a molar excess of a second, labeled antibody directed against the first antibody.

The label used for the detection antibody is any detectable functionality that does not interfere with the binding of the VLP antibodies. Examples of suitable labels are those numerous labels known for use in immunoassay, including moieties that may be detected directly, such as fluorochrome, chemiluminescent, and radioactive labels, as well as moieties, such as enzymes, that must be reacted or derivatized to be detected. Examples of such labels include the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luciferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, biotin/streptavidin, biotin/Streptavidin-β-galactosidase with MUG, spin labels, bacteriophage labels, stable free radicals, and the like. As noted herein, the fluorimetric detection is one example.

Conventional methods are available to bind these labels covalently to proteins or polypeptides. For instance, coupling agents such as dialdehydes, carbodiimides, dimaleimides, bis-imidates, bis-diazotized benzidine, and the like may be used to tag the antibodies with the herein-described fluorescent, chemiluminescent, and enzyme labels. See, for example, U.S. Pat. No. 3,940,475 (fluorimetry) and U.S. Pat. No. 3,645,090 (enzymes); Hunter et al. Nature 144:945 (1962); David et al. Biochemistry 13:1014-1021 (1974); Pain et al. J. Immunol. Methods 40:219-230 (1981); and Nygren J. Histochem. and Cytochem. 30:407-412 (1982). In certain embodiments, labels herein are fluorescent to increase amplification and sensitivity to 8 pg/ml, more preferably biotin with streptavidin-β-galactosidase and MUG for amplifying the signal. In certain embodiments, a colorimetric label is used, e.g., where the detectable antibody is biotinylated and the detection means is avidin or streptavidin-peroxidase and 3,3′,5,5′-tetramethyl benzidine.

The conjugation of such label, including the enzymes, to the antibody is a standard manipulative procedure for one of ordinary skill in immunoassay techniques. See, for example, O'Sullivan et al. “Methods for the Preparation of Enzyme-antibody Conjugates for Use in Enzyme Immunoassay,” in Methods in Enzymology, ed. J. J. Langone and H. Van Vunakis, Vol. 73 (Academic Press, New York, N.Y., 1981), pp. 147-166.

Following the addition of last labeled antibody, the amount of bound antibody is determined by removing excess unbound labeled antibody through washing and then measuring the amount of the attached label using a detection method appropriate to the label, and correlating the measured amount with the amount of VLP in the biological sample.

Detection Methods and Diagnostic Methods

In still further embodiments, the present invention concerns detection methods (e.g., immunodetection methods) for binding, purifying, removing, quantifying and/or otherwise generally detecting biological components such as a ligand (e.g., parvovirus B19) as contemplated by the present invention. The VLP binding agents (e.g., antibodies, monoclonal antibodies, antigen binding fragments, or antibody fragments) prepared in accordance with the present invention may be employed. Some immunodetection methods include immunohistochemistry, flow cytometry, enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle M H and Ben-Zeev O, Methods Mol Biol. 1999; 109:215-37; Gulbis B and Galand P, Hum Pathol. 1993 December; 24(12):1271-85; and De Jager R et al., Semin Nucl Med. 1993 April; 23(2):165-79, each incorporated herein by reference.

In some embodiments, the immunobinding methods include obtaining a sample suspected of comprising a VLP, a parvovirus, an erythrovirus, or a parvovirus B19, and contacting the sample with a first VLP binding agent (e.g., an antibody) in accordance with the present invention, as the case may be, under conditions effective to allow the formation of immunocomplexes.

In some embodiments (and in terms of detection (e.g., antigen detection)), the sample (e.g., biological sample) detected or analyzed may be any sample in which it is desirable to detect a VLP, a parvovirus, an erythrovirus, or a parvovirus B19, such as fluidic extract, blood, plasma, serum, spinal fluid, lymph fluid, tissue section or specimen, homogenized tissue extract, biopsy aspirates, a cell, separated and/or purified forms VLP-containing compositions, parvovirus-containing compositions, erythrovirus-containing compositions, or parvovirus B19-containing compositions, or any biological fluid. In some embodiments, blood, plasma, or lymph samples or extracts are used.

Contacting the chosen sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any ligand protein (e.g., parvovirus B19 capsid proteins) antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In some embodiments, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The anti-ligand antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding agent that has binding affinity for the antibody. In these cases, the second binding agent may be linked to a detectable label. The second binding agent is itself often an antibody, which may thus be termed a “secondary” antibody. The primary immune complexes are contacted with the labeled, secondary binding agent, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding agent, such as an antibody, that has binding affinity for the antibody is used to form secondary immune complexes, as described herein. After washing, the secondary immune complexes are contacted with a third binding agent or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

In another embodiment, a biotinylated monoclonal or polyclonal antibody is used to detect the target antigen(s), and a second step antibody is then used to detect the biotin attached to the complexed biotin. In that method the sample to be tested is first incubated in a solution comprising the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution comprising the second step antibody against biotin. This second step antibody is labeled, as for example with an enzyme that can be used to detect the presence of the antibody/antigen complex by histoenzymology using a chromogen substrate. With suitable amplification, a conjugate can be produced that is macroscopically visible.

Some embodiments of the invention include a method for diagnosis in an animal (e.g., human) with a parvovirus infection, an erythrovirus infection, or a parvovirus B19 infection. In certain embodiments, the method for diagnosis comprising (a) detecting whether parvovirus, erythrovirus, or parvovirus B19 is in a sample from the animal, comprising the method of detecting according any detecting method disclosed herein (e.g., those discussed above), and (b) diagnosing the animal with a parvovirus infection, an erythrovirus infection, or a parvovirus B19 infection, if the presence of parvovirus, erythrovirus, or parvovirus B19 in the sample is detected. In certain embodiments, the method is for diagnosis for a parvovirus infection or a parvovirus B19 infection Animals include but are not limited to mammals, primates, monkeys (e.g., macaque, rhesus macaque, or pig tail macaque), humans, canine, feline, bovine, porcine, avian (e.g., chicken), mice, rabbits, and rats. In other embodiments, the animal is a mammal. In yet other embodiments, the animal is a human or a primate. In still other embodiments, the detection of step (a) uses VLP binding agents (e.g., antibodies, monoclonal antibodies, antigen binding fragments, or antibody fragments). In some embodiments, the detection method of step (a) is an immunodetection methods such as but not limited to immunohistochemistry, flow cytometry, enzyme linked immunosorbent assay (ELISA), radioimmunoassay (RIA), immunoradiometric assay, fluoroimmunoassay, chemiluminescent assay, bioluminescent assay, or Western blot. In certain embodiments, the sample is fluidic extract, blood, plasma, serum, spinal fluid, lymph fluid, tissue section or specimen, homogenized tissue extract, biopsy aspirates, a cell, separated and/or purified forms VLP-containing compositions, or any biological fluid. In some embodiments, the sample is blood, plasma, or lymph samples or extracts.

Compositions and Kits

Also provided by the invention are compositions and kits for use in the practice of the present invention as disclosed herein. Such kits may comprise containers, each with one or more of the various reagents (typically in concentrated form) utilized in the methods, including, for example, one or more binding agents (antibodies), already attached to a marker or optionally with reagents for coupling a binding agent to an antibody (as well as the marker itself), buffers, and/or reagents and instrumentation for the isolation (optionally by microdissection) to support the practice of the invention. A label or indicator describing, or a set of instructions for use of, kit components in a ligand detection method of the present invention, will also be typically included, where the instructions may be associated with a package insert and/or the packaging of the kit or the components thereof.

In still further embodiments, the present invention concerns immunodetection kits for use with the immunodetection methods described herein. As the antibodies are generally used to detect VLPs, the antibodies will generally be included in the kit. The immunodetection kits will thus comprise, in suitable container means, a first antibody that binds to VLPs and/or optionally, an immunodetection reagent and/or further optionally, a VLP or sample containing VLP.

The immunodetection reagents of the kit may take any one of a variety of forms, including those detectable labels that are associated with and/or linked to the given antibody. Detectable labels that are associated with and/or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted herein, a number of exemplary labels are known in the art and/or all such labels may be suitably employed in connection with the present invention.

The kit may further comprise an a VLP detection reagent used to measure parvovirus, erythrovirus, or parvovirus B19 amounts in a subject comprising a VLP detection reagent, and instructions for use. In one embodiment, the VLP detection reagent comprises a VLP binding peptide or anti-VLP antibody. In another embodiment, the kit further comprises a secondary antibody which binds the anti-VLP antibody.

In one embodiment the VLP-specific antibody is included at a concentration of about 0.1 to about 20 μg/mL, about 0.1 to about 15 μg/mL, about 0.1 to about 10 μg/mL, about 0.5 to about 20 μg/mL, about 0.5 to about 15 μg/mL, about 0.5 to about 10 μg/mL, about 1 to about 20 μg/mL, about 1 to about 15 μg/mL, about 1 to about 10 μg/mL, about 2 to about 20 μg/mL, about 2 to about 15 μg/mL, or about 2 to about 10 μg/mL. In another embodiment, the VLP-specific antibody is included at a concentration of about 1.5 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL. In another embodiment, the VLP-specific antibody is included at a concentration of about 2 μg/mL. In another embodiment, the VLP-specific antibody is included at a concentration of about 10 μg/mL.

In another embodiment, the antibody is included in concentrated solution with instructions for dilutions to achieve a final concentration of about 1 to about 20 μg/mL, about 1 to about 15 μg/mL, about 1 to about 10 μg/mL, about 2 to about 20 μg/mL, about 2 to about 15 μg/mL, or about 2 to about 10 μg/mL. In another embodiment, the antibody is included in concentrated solution with instructions for dilutions to achieve a final concentration of about 1.5 μg/mL, about 2 μg/mL, about 3 μg/mL, about 4 μg/mL, about 5 μg/mL, about 6 μg/mL, about 7 μg/mL, about 8 μg/mL, about 9 μg/mL, or about 10 μg/mL. In another embodiment, the antibody is included in concentrated solution with instructions for dilutions to achieve a final concentration of about 2 μg/mL. In another embodiment, the antibody is included in concentrated solution with instructions for dilutions to achieve a final concentration of about 10 μg/ml.

In another embodiment, the kit further comprises a detection reagent selected from the group consisting of: an enzyme, a fluorophore, a radioactive label, and a luminophore. In another embodiment, the detection reagent is selected from the group consisting of: biotin, digoxigenin, fluorescein, tritium, and rhodamine.

The kit can also include instructions for detection and measuring of VLP amount. The kit can also include control or reference samples. Non-limiting examples of control or reference samples include cell pellets or tissue culture cell lines derived from normal (normal control) or a positive control samples. Exemplary cell lines include cell lines stably or transiently transfected with an expression vector that expresses self-assembled VLP. Additional examples include cell pellets and tissue samples.

In some embodiments, a kit is a packaged combination including the basic elements of: (a) capture reagents comprised of the monoclonal antibodies against human VLPs; and (b) detection reagents which can also comprise VLP monoclonal antibodies, but can also comprise detectable (labeled or unlabeled) antibodies that bind to VLP. These basic elements are defined herein.

In one embodiment, the kit further comprises a solid support for the capture reagents, which can be provided as a separate element or on which the capture reagents are already immobilized. Hence, the capture antibodies in the kit can be immobilized on a solid support, or they can be immobilized on such support that is included with the kit or provided separately from the kit.

In one embodiment, the capture reagent is coated on a microtiter plate. The detection reagent can be labeled antibodies detected directly or unlabeled antibodies that are detected by labeled antibodies directed against the unlabeled antibodies raised in a different species. Where the label is an enzyme, the kit will ordinarily include substrates and cofactors required by the enzyme, and where the label is a fluorophore, a dye precursor that provides the detectable chromophore. Where the detection reagent is unlabeled, the kit can further comprise a detection means for the detectable antibodies, such as the labeled antibodies directed to the unlabeled antibodies, e.g., in a fluorimetric-detected format. Where the label is an enzyme, the kit will ordinarily include substrates and cofactors required by the enzyme, where the label is a fluorophore, a dye precursor that provides the detectable chromophore, and where the label is biotin, an avidin such as avidin, streptavidin, or streptavidin conjugated to HRP or β-galactosidase with MUG.

In one embodiment, the capture reagent is the VLP antibody 19, 25, 61, or 91 or an antibody comprising the sequences of antibody 19, 25, 61, or 91. In one embodiment, the detection reagent is the VLP antibody 19, 25, 61, or 91 or an antibody comprising the sequences of antibody 19, 25, 61, or 91. In another embodiment, the detection reagent VLP antibody 19, 25, 61, or 91 or an antibody comprising the sequences of antibody 19, 25, 61, or 91 is biotinylated.

The kit also typically contains instructions for carrying out the assay, and/or VLP, or fragments thereof as an antigen standard, as well as other additives such as stabilizers, washing and incubation buffers, and the like. The kit can also include instructions for detection and scoring of VLP expression.

The components of the kit can be provided in predetermined ratios, with the relative amounts of the various reagents suitably varied to provide for concentrations in solution of the reagents that substantially maximize the sensitivity of the assay. Particularly, the reagents can be provided as dry powders, usually lyophilized, including excipients, which on dissolution will provide for a reagent solution having the appropriate concentration for combining with the sample to be tested.

Compositions comprising the antibodies or antigen binding fragments described herein are also provided. In one embodiment, a composition comprises an anti-VLP antibody or antigen binding fragment described herein and a buffer, e.g., a buffer that can be used in a detection assay such as FACS, IHC, or ELISA. Such buffers are known to those of ordinary skill in the art and include diluents. By way of example, certain FACS buffers are provided herein, e.g., in the working examples. FACS buffers can also contain, for example, serum or albumin (such as calf serum, goat serum, or BSA) and/or sodium azide. FACS buffers can also contain PBS, EDTA, and/or DNAse or any combination thereof. IHC buffers are also provided herein and known to those of ordinary skill in the art. IHC buffers can contain, for example, casein serum or albumin (such as calf serum, goat serum, or BSA), Tween or Triton, PBS and/or sodium azide or any combination thereof. ELISA buffers are also provided herein and known to those of ordinary skill in the art. ELISA buffers can contain, for example, serum or albumin (such as calf serum, goat serum, or BSA), non-fat dry milk, casein, and/or gelatin or any combination thereof.

One or more VLP binding agents (e.g., antibodies, monoclonal antibodies, antigen binding fragments, or antibody fragments) can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

One or more VLP binding agents (e.g., antibodies, monoclonal antibodies, antigen binding fragments, or antibody fragments) can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%. Some embodiments of the present invention include compositions comprising one or more VLP binding agents (e.g., antibodies, monoclonal antibodies, antigen binding fragments, or antibody fragments). In certain embodiments, the composition is a pharmaceutical composition (e.g., a vaccine), such as compositions that are suitable for administration to animals (e.g., mammals, primates, monkeys, humans, canine, porcine, mice, rabbits, or rats).

Some embodiments of the present invention include compositions comprising one or more VLP binding agents (e.g., antibodies, monoclonal antibodies, antigen binding fragments, or antibody fragments). In certain embodiments, the composition is a pharmaceutical composition (e.g., a vaccine), such as compositions that are suitable for administration to animals (e.g., mammals, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

“Therapeutically effective amount” means an amount effective to achieve a desired and/or beneficial effect. An effective amount can be administered in one or more administrations. For some purposes of this invention, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any method known in the art, such as measurement of antibody titers.

In some embodiments, one or more VLP binding agents (e.g., antibodies, monoclonal antibodies, antigen binding fragments, or antibody fragments) can be part of a pharmaceutical composition (e.g., a vaccine) and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof.

In certain embodiments, pharmaceutical compositions can be formulated to release the active ingredient (e.g., one or more VLP binding agents (e.g., antibodies, monoclonal antibodies, antigen binding fragments, or antibody fragments)) substantially immediately upon the administration or any substantially predetermined time or time after administration. Such formulations can include, for example, controlled release formulations such as various controlled release compositions and coatings.

Other formulations (e.g., formulations of a pharmaceutical composition) can, in certain embodiments, include those incorporating the drug (or control release formulation) into food, food stuffs, feed, or drink.

Other embodiments of the invention can include methods of administering or treating an organism, which can involve treatment with an amount of at least one VLP binding agent (e.g., antibodies, monoclonal antibodies, antigen binding fragments, or antibody fragments) that is effective to treat the disease, condition, or disorder that the organism has, or is suspected of having, or is susceptible to, or to bring about a desired physiological effect. In some embodiments, the composition or pharmaceutical composition comprises at least one VLP binding agent (e.g., antibodies, monoclonal antibodies, antigen binding fragments, or antibody fragments) which can be administered to an animal (e.g., mammals, primates, monkeys, or humans) in an amount of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some animals (e.g., mammals, mice, rabbits, feline, porcine, or canine) can be administered a dosage of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, those skilled in the art will appreciate that it is possible to employ many concentrations in the methods of the present invention, and using, in part, the guidance provided herein, will be able to adjust and test any number of concentrations in order to find one that achieves the desired result in a given circumstance. In other embodiments, the compounds of the invention can be administered in combination with one or more other therapeutic agents for a given disease, condition, or disorder.

In some embodiments, the compositions can include a unit dose of one or more VLP binding agents (e.g., antibodies, monoclonal antibodies, antigen binding fragments, or antibody fragments) in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

In certain embodiments, compositions (e.g., pharmaceutical compositions or vaccines) can include one or more adjuvants. In some embodiments, adjuvants are not included in the composition. In still other embodiments, the composition comprises one or more adjuvants, such as, but not limited to polymers of acrylic or methacrylic acid, maleic anhydride and alkenyl derivative polymers; immunostimulating sequences (ISS), an oil in water emulsion (e.g., the SPT emulsion described on p 147 of “Vaccine Design, The Subunit and Adjuvant Approach” published by M. Powell, M. Newman, Plenum Press 1995, and the emulsion MF59 described on p 183 of the same reference), cation lipids containing a quaternary ammonium salt, A1K(SO₄)₂, AlNa(SO₄)₂, AlNH(SO₄)₄, silica, alum, AI(OH)₃, Ca₃(PO₄)₂, kaolin, carbon, aluminum hydroxide, muramyl dipeptides, N-acetyl-muramyl-L-threonyl-D-isoglutamine (thr-DMP), N-acetyl-nornuramyl-L-alanyl-D-isoglutamine (CGP 11687, also referred to as nor-MDP), N-acetylmuramyl-L-alanyl-D-isoglutaminyl-L-alanine-2-(1′2′-dipalmitoyl-s-n-glycero-3-hydroxphosphoryloxy)-ethylamine (CGP 19835A, also referred to as MTP-PE), RIBI (MPL+TDM+CWS) in a 2% squalene/Tween-80® emulsion, lipopolysaccharides and its various derivatives, including lipid A, Freund's Complete Adjuvant (FCA), Freund's Incomplete Adjuvants, Merck Adjuvant 65, polynucleotides (for example, poly IC and poly AU acids), wax D from Mycobacterium, tuberculosis, substances found in Corynebacterium parvum, Bordetella pertussis, and members of the genus Brucella, liposomes or other lipid emulsions, ISCOMS, Quil A, ALUN, Lipid A derivatives, choleratoxin derivatives, HSP derivatives, LPS derivatives, synthetic peptide matrixes or GMDP, cytokines, Interleukin 1, Interleukin 2, Montanide ISA-51, QS-21, TITERMAX®, or ADJUPLEX™ Vaccine Adjuvant.

In some embodiments, additional adjuvants or compounds that can be used (e.g., to modify or stimulate the immune response) include ligands for Toll-like receptors (TLRs). In mammals, TLRs are a family of receptors expressed on DCs that recognize and respond to molecular patterns associated with microbial pathogens. Several TLR ligands have been intensively investigated as vaccine adjuvants. Bacterial lipopolysaccharide (LPS) is the TLR4 ligand and its detoxified variant mono-phosphoryl lipid A (MPL) is an approved adjuvant for use in humans. TLR5 is expressed on monocytes and DCs and responds to flagellin whereas TLR9 recognizes bacterial DNA containing CpG motifs. Oligonucleotides (OLGs) containing CpG motifs are potent ligands for, and agonists of, TLR9 and have been intensively investigated for their adjuvant properties. In some embodiments, the adjuvant is alum. In some embodiments the adjuvant is not M59 adjuvant.

Parenteral administration, if used, is generally characterized by injection. Sterile injectables can be prepared in conventional forms, either as liquid solutions or suspensions, solid forms suitable for solution or suspension in liquid prior to injection, or as emulsions.

Administration Routes and Treatments of Disease

The VLP binding agents (e.g., antibodies, monoclonal antibodies, antigen binding fragments, or antibody fragments) of the invention or mVLPs can be administered to animals by any number of suitable administration routes or formulations. The VLP binding agents (e.g., antibodies, monoclonal antibodies, antigen binding fragments, or antibody fragments) of the invention or mVLPs can also be used to treat animals for a variety of diseases Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects. A subject susceptible to a parvovirus infection, an erthrovirus infection, or a B19 parvovirus (e.g., human) infection can be a human or an animal subject.

The route of administration of the VLP binding agents (e.g., antibodies, monoclonal antibodies, antigen binding fragments, or antibody fragments) of the invention or mVLPs can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, subcutaneous administration, topical administration, intradermal administration, oral administration, sublingual administration, intranasal administration, or intramuscular administration. The choice of administration route can depend on the VLP binding agent (e.g., antibodies, monoclonal antibodies, antigen binding fragments, or antibody fragments) identity (e.g., the physical and chemical properties of the VLP binding agent) or mVLP identity (e.g., the physical and chemical properties of the mVLP) as well as the age and weight of the animal, the particular disease, and the severity of the disease. Of course, combinations of administration routes can be administered, as desired.

Some embodiments of the invention include a method for providing a subject with a composition comprising a VLP binding agent (e.g., antibodies, monoclonal antibodies, antigen binding fragments, or antibody fragments) and/or mVLPs described herein (e.g., a pharmaceutical composition) which comprises one or more administrations of one or more such compositions; the compositions may be the same or different if there is more than one administration.

Diseases that can be treated in an animal (e.g., mammals, porcine, canine, avian (e.g., chicken), bovine, feline, primates, monkeys, rabbits, and humans) using the VLP binding agents (e.g., antibodies, monoclonal antibodies, antigen binding fragments, or antibody fragments) and/or mVLPs include, but are not limited to parvovirus infections, diseases related to parvovirus infections, erythrovirus infections, diseases related to erythrovirus infections, parvovirus B19 infections, and diseases related to parvovirus B19 infection. Some diseases related to parvovirus infections (e.g., erythrovirus infections or parvovirus B19 infections) include, but are not limited to, hydrops fetalis intrauterine fetal death, erythema infectiosum (i.e., fifth disease), hereditary diseases (e.g., sickle cell anemia or Thalassemia), acquired diseases (e.g., anemia or anemia induced by malaria), parvovirus B19-induced red cell aplasia (TRCA), chronic anemia, diseases related to immunodeficient individuals (e.g., recipients of organ transplants, animals undergoing chemotherapy, animals undergoing bone marrow transplant, or HIV-positive animals), acute arthropathy, persistent arthropathy, aplastic crisis, arthritis, hepatitis, myocarditis, hepatosplenomegaly, acute thyroiditis, subacute thyroiditis, Graves' disease, Hashimoto's thyroiditis, and autoimmune diseases (e.g., autoimmune thyroid diseases, systemic lupus erythematosus (SLE), meningiencephalitis, or fibromyalgia). Other diseases related to parvovirus infections (e.g., erythrovirus infections or parvovirus B19 infections) include, but are not limited to, gastrointestinal tract damage, dehydration, cardiac syndrome, lethargy, diarrhea (e.g., severe diarrhea), fever, vomiting, loss of appetite, stillbirth, mummification, embryonic death, infertility, low white blood cell count, cerebellar hypoplasia, lymphadenopathy, splenomegaly, glomerulonephritis, and anemia Animals that can be treated include but are not limited to mammals, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. A subject susceptible to a parvovirus infection, an erthrovirus infection, or a B19 parvovirus (e.g., human) infection can be a human or animal subject. In some instances, the animal is in need of the treatment (e.g., a prophylactic treatment).

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating a parvovirus infection (e.g., an erythrovirus infection or a parvovirus B19 infection), treating can include but is not limited to prophylactic treatment and therapeutic treatment. As such, treatment can include, but is not limited to: conferring protection against a parvovirus infection (e.g., an erythrovirus infection or a parvovirus B19 infection); preventing a parvovirus infection (e.g., an erythrovirus infection or a parvovirus B19 infection); reducing the risk of parvovirus infection (e.g., an erythrovirus infection or a parvovirus B19 infection); ameliorating or relieving symptoms of a parvovirus infection (e.g., an erythrovirus infection or a parvovirus B19 infection); eliciting an immune response against a parvovirus (e.g., an erythrovirus or a parvovirus B19) or an antigenic component thereof; inhibiting the development or progression of a parvovirus infection (an erythrovirus infection or a parvovirus B19 infection); inhibiting or preventing the onset of symptoms associated with a parvovirus infection (e.g., an erythrovirus infection or a parvovirus B19 infection); reducing the severity of a parvovirus infection (e.g., an erythrovirus infection or a parvovirus B19 infection); and causing a regression of a parvovirus infection (e.g., an erythrovirus infection or a parvovirus B19 infection) or one or more of the symptoms associated with a parvovirus infection (e.g., an erythrovirus infection or a parvovirus B19 infection). In some embodiments, treating does not include prophylactic treatment (e.g., vaccination or otherwise preventing or ameliorating future disease).

Symptoms associated with parvovirus infection (e.g., an erythrovirus infection or a parvovirus B19 infection) are known to those of ordinary skill in the art and can include those described herein and well-known to those of ordinary skill in the art. The presence of an infection can be assessed using methods known to those or ordinary skill in the art. In some cases, the presence of a parvovirus infection (e.g., an erythrovirus infection or a parvovirus B19 infection) can be determined using methods known to those of ordinary skill in the art.

Treatment of an animal can occur using any suitable administration method (such as those disclosed herein) and using any suitable amount of VLP binding agents (e.g., antibodies, monoclonal antibodies, antigen binding fragments, or antibody fragments) (such as those disclosed herein) or any suitable amount of mVLP (such as those disclosed herein). In some embodiments, methods of treatment comprise treating an animal for a parvovirus infection (e.g., in a human or primate), a disease related to a parvovirus infection (e.g., in a human or primate), an erythrovirus infection (e.g., in a human or primate), a disease related to an erythrovirus infection (e.g., in a human or primate), a disease related to a parvovirus B19 infection (e.g., in a human or primate), a parvovirus B19 infection (e.g., in a human or primate), or combinations thereof. Some embodiments of the invention include a method for treating a subject (e.g., an animal such as a human or primate) with a composition (e.g., a pharmaceutical composition) comprising a VLP binding agent (e.g., antibodies, monoclonal antibodies, antigen binding fragments, or antibody fragments) described herein and/or an mVLP described herein, which comprises one or more administrations of one or more such compositions; the compositions may be the same or different if there is more than one administration. Other embodiments of the invention include a method for inducing an immune response in an animal, comprising one or more administrations of one or more compositions (e.g., a pharmaceutical composition) comprising one or more VLP binding agents (e.g., antibodies, monoclonal antibodies, antigen binding fragments, or antibody fragments) described herein and/or an mVLP described herein.

Other embodiments of the invention include a method for treating an animal for a parvovirus infection, a disease related to a parvovirus infection, an erythrovirus infection, a disease related to an erythrovirus infection, a parvovirus B19 infection, or a disease related to a parvovirus B19 infection, comprising (a) detecting whether parvovirus, erythrovirus, or parvovirus B19 is in a sample from the animal, comprising the method of detecting according to any detection method described herein, and (b) administering one or more administrations of one or more compositions comprising one or more of an mVLP, a VLP binding agent of any VLP binding agent described herein, or an antibiotic, if the presence of parvovirus, erythrovirus, or parvovirus B19 in the sample is detected. In some embodiments, the detection method of step (a) is an immunodetection method such as but not limited to immunohistochemistry, flow cytometry, enzyme linked immunosorbent assay (ELISA), radioimmunoassay (RIA), immunoradiometric assay, fluoroimmunoassay, chemiluminescent assay, bioluminescent assay, or Western blot. In certain embodiments, the sample is fluidic extract, blood, plasma, serum, spinal fluid, lymph fluid, tissue section or specimen, homogenized tissue extract, biopsy aspirates, a cell, separated and/or purified forms VLP-containing compositions, or any biological fluid. In some embodiments, the sample is blood, plasma, or lymph samples or extracts. In other embodiments, the antibiotic comprises ampicillin, a cephalexin, or a flouroquinolone, or combinations thereof. In still other embodiments, the mVLP comprises a VP2 polypeptide with at least one amino acid modification relative to a wild type VP2 and the wild type VP2 has the amino acid sequence of SEQ ID NO: 1. In yet other embodiments, (a) the mVLP comprises a VP2 polypeptide with at least one amino acid modification relative to a wild type VP2, (b) the wild type VP2 has the amino acid sequence of SEQ ID NO: 1, and (c) the at least one amino acid modification (1) comprises (i) Y401F and (ii) Q399N or Q404T, (2) is Y401F, (3) is Q368A and Q369A, (4) is Q399N, Q400N, and Q404T, or (5) is Y392A. In some embodiments, the VP2 polypeptide is not construct J. In still other embodiments, the VP2 polypeptide sequence has at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity to SEQ ID NO: 1. In yet other embodiments, the at least one amino acid modification comprises (a) Y401F and (b) Q399N or Q404T. In some embodiments, the VP2 polypeptide is selected from the group consisting of construct A, construct D, construct F, construct G, and construct H. In other embodiments, is Construct F. In yet other embodiments, the mVLP comprises a VP2 that has at least one amino acid modification (1) comprising (a) Y401F and (b) Q399N or Q404T or (2) is Y401F, and the VP2 polypeptide is not construct J.

Certain embodiments of the invention include a method for inducing an immune response in an animal comprising (a) detecting whether parvovirus, erythrovirus, or parvovirus B19 is in a sample from the animal, comprising the method of detecting according to any detection method described herein, and (b) administering one or more administrations of one or more compositions comprising one or more of an mVLP, a VLP binding agent of any VLP binding agent described herein, or an antibiotic, if the presence of parvovirus, erythrovirus, or parvovirus B19 in the sample is detected. In some embodiments, the detection method of step (a) is an immunodetection method such as but not limited to immunohistochemistry, flow cytometry, enzyme linked immunosorbent assay (ELISA), radioimmunoassay (RIA), immunoradiometric assay, fluoroimmunoassay, chemiluminescent assay, bioluminescent assay, or Western blot. In certain embodiments, the sample is fluidic extract, blood, plasma, serum, spinal fluid, lymph fluid, tissue section or specimen, homogenized tissue extract, biopsy aspirates, a cell, separated and/or purified forms VLP-containing compositions, or any biological fluid. In some embodiments, the sample is blood, plasma, or lymph samples or extracts. In other embodiments, the antibiotic comprises ampicillin, a cephalexin, or a flouroquinolone, or combinations thereof. In still other embodiments, the mVLP comprises a VP2 polypeptide with at least one amino acid modification relative to a wild type VP2 and the wild type VP2 has the amino acid sequence of SEQ ID NO: 1. In yet other embodiments, (a) the mVLP comprises a VP2 polypeptide with at least one amino acid modification relative to a wild type VP2, (b) the wild type VP2 has the amino acid sequence of SEQ ID NO: 1, and (c) the at least one amino acid modification (1) comprises (i) Y401F and (ii) Q399N or Q404T, (2) is Y401F, (3) is Q368A and Q369A, (4) is Q399N, Q400N, and Q404T, or (5) is Y392A. In some embodiments, the VP2 polypeptide is not construct J. In still other embodiments, the VP2 polypeptide sequence has at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, at least about 99% identity to SEQ ID NO: 1. In yet other embodiments, the at least one amino acid modification comprises (a) Y401F and (b) Q399N or Q404T. In some embodiments, the VP2 polypeptide is selected from the group consisting of construct A, construct D, construct F, construct G, and construct H. In other embodiments, is Construct F. In yet other embodiments, the mVLP comprises a VP2 that has at least one amino acid modification (1) comprising (a) Y401F and (b) Q399N or Q404T or (2) is Y401F, and the VP2 polypeptide is not construct J.

In some embodiments, the method of treatment includes administering an effective amount of a composition (e.g., pharmaceutical composition) comprising a VLP binding agent (e.g., antibodies, monoclonal antibodies, antigen binding fragments, or antibody fragments) and/or an mVLP. As used herein, the term “effective amount” refers to a dosage or a series of dosages sufficient to affect treatment (e.g., to treat a parvovirus infection such as an erythrovirus infection or a parvovirus B19 infection or to treat diseases related to a parvovirus infection such as diseases related to an erythrovirus infection or diseases related to a parvovirus B19 infection) in an animal. In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein.

In some embodiments, an effective amount of at least one VLP binding agent (e.g., antibodies, monoclonal antibodies, antigen binding fragments, or antibody fragments) (which can be administered to an animal such as mammals, primates, monkeys or humans) can be an amount of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some animals (e.g., mammals, mice, rabbits, feline, porcine, or canine) can be administered a dosage of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg.

In some embodiments, an effective amount of at least one mVLP (which can be administered to an animal such as mammals, primates, monkeys or humans) can be an amount of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight or about 6.5 mg/kg human body weight. In some instances, some animals (e.g., mammals, mice, rabbits, feline, porcine, or canine) can be administered a dosage of about 0.01 to about 15 mg/kg body weight, about 0.1 to about 10 mg/kg body weight, about 0.5 to about 7 mg/kg body weight, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg.

As used herein, “immune response” refers to a response by the immune system of a subject. For example, immune responses include, but are not limited to, a detectable alteration (e.g., increase) in Toll receptor activation, lymphokine (e.g., cytokine (e.g., Th1 or Th2 type cytokines) or chemokine) expression and/or secretion, macrophage activation, dendritic cell activation, T cell activation (e.g., CD4+ or CD8+ T cells), NK cell activation, and/or B cell activation (e.g., antibody generation and/or secretion). Additional examples of immune responses include binding of an immunogen to an MHC molecule and inducing a cytotoxic T lymphocyte (“CTL”) response, inducing a B cell response (e.g., antibody production), and/or T-helper lymphocyte response, and/or a delayed type hypersensitivity (DTH) response against the antigen from which the immunogenic polypeptide is derived, expansion (e.g., growth of a population of cells) of cells of the immune system (e.g., T cells, B cells (e.g., of any stage of development (e.g., plasma cells))), and increased processing and presentation of antigen by antigen presenting cells. An immune response can be to immunogens that the subject's immune system recognizes as foreign (e.g., non-self antigens from microorganisms (e.g., pathogens), or self-antigens recognized as foreign). Thus, it is to be understood that, as used herein, “immune response” refers to any type of immune response, including, but not limited to, innate immune responses (e.g., activation of Toll receptor signaling cascade and/or activation of complement), cell-mediated immune responses (e.g., responses mediated by T cells (e.g., antigen-specific T cells) and non-specific cells of the immune system), and humoral immune responses (e.g., responses mediated by B cells (e.g., via generation and secretion of antibodies into the plasma, lymph, and/or tissue fluids)). The term “immune response” is meant to encompass all aspects of the capability of a subject's immune system to respond to antigens and/or immunogens (e.g., both the initial response to an immunogen (e.g., a pathogen) as well as acquired (e.g., memory) responses that are a result of an adaptive immune response).

Up to 85% of the adult population is sero-positive for Parvovirus B19 infection. Parvovirus B19 infection can cause hydrops fetalis and intrauterine fetal death, although it is most widely known to be related to erythema infectiosum (fifth disease) and can be asymptomatic in healthy individuals. Older children and adults with either hereditary (sickle cell anemia) or acquired (anemia induced by malaria) anemia are at risk for developing parvovirus B19-induced red cell aplasia (TRCA) or death. The cause of the chronic anemia in immunodeficient individuals, such as recipients of organ transplants or HIV-positive patients, was contributed to parvovirus B19 infection. In some instances, the pathological manifestations of parvovirus B19 infection can be affected by the patient's immunologic and hematologic status, and can induce more severe disease, such as acute or persistent arthropathy, aplastic crisis, and also been implicated in arthritis, hepatitis, myocarditis, hepatosplenomegaly, a spectrum of autoimmune diseases such as systemic lupus erythematosus (SLE), meningiencephalitis, or fibromyalgia.

In some embodiments, children (i.e., ages from about 0 to about 18) can be vaccinated before they enter elementary school (i.e., ages from about 0 to about 13). In other embodiments, immunization can be administered to animals at risk. Animals at risk include but are not limited to animals that have had a transfusion, an organ transplant, animals with infectious disease (e.g., HIV or malaria), pregnant animals (e.g., human women) with children (e.g., under the age of 18), animals infected by parvovirus B19 infection, animals (e.g., children) living in an area where malaria is prevalent, immunodeficient animals (e.g., recipients of organ transplants, animals undergoing chemotherapy, animals undergoing bone marrow transplant, or HIV-positive animals), or animals with an autoimmune disease (e.g., systemic lupus erythematosus; SLE, meningiencephalitis and fibromyalgia).

In some embodiments, the treatments disclosed herein can include use of other drugs (e.g., antibiotics) or therapies for treating disease. For example, other antibiotics or mVLPs can be used to treat infections and can be combined with a VLP binding agent (e.g., antibodies, monoclonal antibodies, antigen binding fragments, or antibody fragments) to treat disease (e.g., infections). In other embodiments, intravenous immunoglobulin (IVIG) therapy can be used as part of the treatment regime (i.e., in addition to administration of VLP binding agents (e.g., antibodies, monoclonal antibodies, antigen binding fragments, or antibody fragments)) of parvovirus infection (e.g., erythrovirus infection or parvovirus B19 infection).

The presently-disclosed subject matter is further illustrated by the following specific but non-limiting examples. The following examples may include compilations of data that are representative of data gathered at various times during the course of development and experimentation related to the present invention.

### EXAMPLES

Some aspects of the Examples are related to WO 2015/138424 A1, published Sep. 17, 2015, which is herein incorporated by reference in its entirety.

### Materials and Methods

TABLE AA
B19 VP2 Polypeptide Wild Type and Mutants

| Construct | | Amino acids |
|---|---|---|
| designation: | Mutation | (s) mutated |
| B19 Wild Type | none | none |
| (SEQ ID NO: 1) | | |
| A | QQYTDQ to QQFTDQ | Y401F |
| B | QQYTDQ to QQWTDQ | Y401W |
| D | KEYQQ to KEYAA | Q368A and Q369A |
| F | QQYTDQ to NNFTDT | Q399N, Q400N, |
| | | Y401F, and Q404T |

Production of Virus-Like Particles (VLPs)

VLPs were extracted from Sf9 insect cells infected with recombinant baculovirus (Autographa californica nucleopolyhedroviruses) expressing parvovirus B19 VP2 proteins that were purified on a CsCl continuous gradient. Recombinant baculoviruses expressing mutated VP2 or WT VP2 were generated by PCR cloning of VP2 genes to baculovirus transfer vector. Firstly, minigenes were synthesized in vitro (Integrated DNA technologies or GenScript, IA; Genbank accession number: NC_000883.2, AY044266.2 [LaLi strain: genotype 2], AJ249437.1 [V9 strain]), and employed as the template of PCRs. For mVLPs, mutations were generated by double PCR. VP2 genes were cloned into a baculovirus transfer vector followed by recombination with baculovirus DNA using a kit (Bac-to-Bac baculovirus expression system, ThermoFisher Scientific/Life Technologies, CA). For purification of VLPs, cells infected by recombinant baculovirus were collected at 72 to 96 hour of post-infection, suspended in Tris buffer (20 mM Tris, 0.25 M NaCl, pH 8.5), downs homogenized, and incubated for 10 min at 45° C. The pH and density of samples were adjusted to 7.2 and 1.30 g/ml, respectively, and centrifuged overnight (32,000 rpm in SW55Ti: Beckman Coulter, MO). Bands containing VLPs were collected, dialyzed against Dulbecco's Phosphate-Buffered Saline (DPBS) (Invitrogen, CA), and examined for their protein concentration using Bio-Rad Bradford protein assay reagent (Bio-Rad Laboratories, USA). The structure of the VLPs was observed under the electron microscope (Phillips CM-120), and their antigenic reactivity was measured with rabbit polyclonal antibodies (PAB) against VP2-VLPs.

Rabbit Polyclonal Antibodies

Rabbit polyclonal antibodies were generated in New Zealand white rabbits by three immunizations with the wild-type genotype B19V, the F₄₀₁-mutated VLPs, and N₃₉₉N₄₀₀F₄₀₁TDT₄₀₄-mutated VLPs at 10 μg per injection containing either TITERMAX® (Sigma-Aldrich) or Sigma Adjuvant System (Sigma-Aldrich). These antibodies were highly reactive with both WT VLPs and mutated VLPs (See Table A1).

ELISA

First, 96-well ELISA plates (Immunolon 2; Dynatech) were coated, either 1 h at 37° C. or overnight at 4° C., with 250 to 500 ng per well of B19V VLPs. Intact VLPs were re-suspended in PBS, and the disrupted VLPs were re-suspended in the following solution: 0.2 M NaCO₃, 0.01 M dithiothreitol, pH 7.0, to detect sequential epitopes. The plates were washed three times with PBS, then saturated with bovine albumin via PBS containing 5% bovine serum albumin (5% PBSA) for 1 h at 37° C. After washing three times, plates were then incubated with primary antibodies. Primary antibodies used were rabbit sera (at 1/1000), mouse sera (1/100 dilution) to B19V VLPs, or supernatant of hybridoma cells without dilution for 1 h at 37° C. Thereafter, plates were washed thrice with PBS, then incubated with alkaline phosphatase-conjugated secondary antibodies, with either goat anti-mouse or anti-rabbit IgG (H+L) (Sigma, USA) at a dilution of 1:5000 for 1 h at 37° C. After washing with PBS, bound antibodies were detected with an alkaline phosphatase chromogenic substrate (Phosphatase substrate, Sigma, USA), and absorption was measured at 405 nm. One percent PBSA and sera collected from pre-immune animals were used as negative controls. Human papillomavirus (HPV) and mouse parvovirus (MPV) VLPs were used to check the cross-reactivity of primary antibodies with other types of VLPs. All antibodies were diluted in 1% PBSA.

Sodium Dodecyl Sulfate-Polyacrylamide Gel Electrophoresis (SDS-PAGE) and Immunoblot (IB) Analysis

The reactivity of Monoclonal Antibodies (MAbs) with denatured VP2 proteins was examined by IB. First, 0.25 μg of purified VLPs were mixed with SDS loading buffer (20% glycerol, 4% SDS, 100 mM Tris, pH 6.8, 0.002% bromophenol blue), and heated at 95° C. for 5 min. Proteins were separated by SDS-PAGE in NUPAGE 4-12% Bis-TRIS gels (Invitrogen, USA). After migration of the proteins, gels were either stained with Coomassie blue (SimplyBlue SafeStain; Invitrogen, USA) or processed for IB. For IB, the separated proteins were transferred to a PVDF membrane (Immobilon-P transfer membrane, EMD Millipore, USA) followed by saturation with 5% milk solution in TBST (0.02 mM Tris, 0.15 M NaCl, 0.1% Tween 20) for 1 h. The membranes were then incubated overnight at 4° C. with primary antibodies to B19V diluted in TBST containing 1% skim milk. The membranes were washed three times with TBST, incubated with 1:3000 diluted secondary antibody (peroxidase conjugated goat anti-mouse IgG, Thermo Scientific Pierce, USA) for 1 h, developed with chemiluminescence substrate (SuperSignal West Dura Extended Duration Substrate; Thermo Scientific Pierce, USA), and visualized on X-ray film (CL-Xposure Film, Thermo Scientific Pierce, USA) using an SRX-101A processor (Konica Minolta Medical Imaging USA, Inc). B19V antibody MAB8293 (EMD Millipore Corporation, MA), which is specific to amino acid residues 328-344 of VP2, and hyperimmune sera raised against WT VP2-VLPs were employed as control positive antibodies.

Hemagglutination Inhibition Assay (HIA)

First, 100 μl of hybridoma cell culture supernatant was mixed with 50 μl (concentration of 1 μg/μl) of WT VLP solution in PBS (0.05 M, pH 6.3) containing 0.2% bovine serum albumin and 0.5% dextrose. This mixture was placed in a round-bottom 96-well plate, incubated for 30 mM at 37° C., and then 50 μl of 4% human red blood cells (RBCs: 0 type human blood; Innovative Research, MI) were added for at least another 5 h of incubation. DPBS (Life Technologies, Inc, CA, USA) was used as a negative control.

Immunization of Mice

BALB/c mice (Jackson Laboratory, Bar Harbor, Me.) were intraperitoneally immunized three times with purified VLPs at 5 to 50 μg dose per mouse using MPL®+TDM adjuvant (Sigma-Aldrich, USA) or Sigma adjuvant system (Sigma-Aldrich, USA) at two week interval between first and second immunizations, as well as a four to six week interval between second and third immunizations. After the second immunization, mice were intravenously bled from their cheek for less than 100 μl of blood in order to examine their immune responses.

Generation of Hybridoma Cells for the Production of Monoclonal Antibodies

Cell fusions to obtain hybridoma cells were carried out using a well-established protocol. Briefly, three days prior to fusion, mice were given a boost immunization, and their spleens were collected aseptically. In some cases, in vitro boosts were employed to enhance the efficacy of hybridization. For in vitro immunization, 20 μg/ml of MDP (N-acethymuramyl-L-alanyl-D-isoglutamine) and approximately 1 μg immunogen was added to the cell culture medium, and then cells were incubated for five days. Splenic cells containing B-cell lymphoblast, were spread it out with 18 gauge needles, washed with incomplete RPMI 1640, and fused with Sp2/O-Ag14 murine myeloma cells (ATCC CRL-1581™; American type culture collection, VA, USA) using Hybri-Max PEG/DMSO solution (Sigma-Aldrich, USA). Fused cells were suspended in high glucose RPMI 1640 (Life Technologies, Inc or Sigma-Aldrich, USA) containing 20% fetal bovine serum (FBS: HyClone fetal bovine serum, Thermo Fisher Scientific, USA) and cultured overnight in a flat bottom 96-well plate. To select for the fused cells, culture medium was replaced with RPMI containing 10% FBS and 1×HAT (hypoxanthine-aminopterin-thymidine: Sigma-Aldrich, USA) for three days, and 1×HT (hypoxanthine-thymidine:Sigma-Aldrich, USA) afterwards. The presence of antibodies was examined by ELISA. Cells that were positive for secretion of the B19V antibody were cloned by the limited dilution method as previously described. MAbs from hybridoma cells were characterized by ELISA, IB and HIA. The resulting hybridoma cell lines were frozen to −150° C. using RPMI with 50% FBS and 10% DMSO as the freezing medium.

### Results

VLPs can mimic the surface epitopes on virion capsids, and are vaccine candidates for viruses. Unfortunately, VLPs of parvovirus B19 were non immunogenic in mice (TABLE A1) resulting in an inability to generate murine MAbs, that are needed to study the immunogenicity of VLPs and infectious virions of parvovirus B19. We were able to increase the immunogenicity of VLPs in mice by eliminating its hemagglutination activities, and eventually generated murine MAbs that reacted with both our mutated hemagglutination negative VLPs and wt VLPs. The immunogenicity of non-mutated VLPs (WT VLPs of genotype 1) was low in BALB/c mice, and all attempts to obtain hybridoma cells secreting MAbs to intact wtVLPs failed. In contrast to the wtVLPs of B19V, we used the mutated VLPs to generate 10 hybridoma cell lines that secreted antibodies specific for VLPs of VP2 VLPs (Table A2).

TABLE A1
Immunoreactions of purified VLPs of non-mutated and
mutated VLPs with rabbit polyclonal antibodies (RPAB)
and murine polyclonal antibodies (MPAB) by ELISA.

| | Polyclonal Antibodies (PAB) | | | | |
|---|---|---|---|---|---|
| | RPAB* | RPAB* | MPAB* | MPAB* | MPAB* |
| | B19V | F₄₀₁- | B19V | F₄₀₁- | NNF₄₀₁TDT- |
| Antigen (VLPs) | genotype 1 | B19V | genotype 1 | B19V | B19V |
| ¹B19V genotype 1 | 1.087 | 2.556 | 0.237 | 1.449 | 1.067 |
| F₄₀₁- B19V | 0.936 | 2.124 | 0.267 | 1.373 | 1.254 |
| NNF₄₀₁TDT-B19V | 1.351 | 3.487 | 0.597 | 2.187 | 1.709 |
| ²B19V genotype 2 | 1.029 | 2.440 | 0.124 | 0.971 | 0.830 |
| ³B19V genotype 3 | 1.186 | 2.533 | 0.258 | 1.564 | 1.200 |

¹Genotype 1 VLPs made from VP2 with sequence ID NO: 1
²Genotype 2 VLPs made from VP2 with sequence ID NO: 53
³Genotype 3 VLPs made from VP2 with sequence ID NO: 54
*B19V: parvovirus B19

TABLE A2
Characterization of MAbs against mutated VP2 VLPs of parvovirus B19

| | | VLP antigens coated on ELISA plates | | | | | | |
|---|---|---|---|---|---|---|---|---|
| | | Prototype | | | | | Disrupted | |
| Antigen | MAbs | Q₃₉₉QYTDQ₄₀₄ | NNF₄₀₁TDT | F₄₀₁ | **KEYA₃₆₈A₃₆₉ | **QQW₄₀₁TDQ | NNF₄₀₁TDT | **HPV16 |
| NNF₄₀₁TDT | 19A* | 2.5 | 3.206 | 3.888 | 0.995 | 1.045 | 0.003 | 0.014 |
| | 19B* | 1.025 | 2.16 | 1.443 | 0.375 | 0.457 | 0.003 | 0.015 |
| | 21 | 2.644 | 2.824 | 2.997 | 1.789 | 1.168 | 1.008 | 0.011 |
| | 25 | 2.86 | 3.262 | 3.817 | 2.789 | 1.529 | 2.113 | 0.009 |
| | 37 | 3.655 | 3.932 | 3.856 | 2.979 | 2.405 | 2.414 | 0.014 |
| | 41 | 3.861 | 3.907 | 3.887 | 2.826 | 2.074 | 3.742 | 0.008 |
| | 51B | 3.608 | 3.874 | 3.666 | 1.738 | 1.815 | 0.779 | 0.005 |
| | 91 | 3.721 | 3.863 | 3.009 | 2.587 | 1.643 | 0.708 | 0.005 |
| | 61* | 2.949 | 3.737 | 3.932 | 2.27 | 1.164 | 0.007 | 0.061 |
| F₄₀₁ | 12* | 0.028 | 0.017 | 3.86 | 0.007 | 0.01 | 0.001 | 0.007 |

(*) indicates hybridoma cell lines against conformational epitopes
(**): KEYA₃₆₈A₃₆₉ and QQW₄₀₁TDQ-VLPs of B19V and HPV16-VLPs were generated previously for other studies (WHITE et al. (1999) “Characterization of a major neutralizing epitope on human papillomavirus type 16 Li” J Virol., Vol. 73, No. 6, pp. 4882-4889.)
ELISA values in bold shows reactivity against conformational epitopes

Out of these, nine cell lines were generated against N₃₉₉N₄₀₀F₄₀₁TDT₄₀₄-VLPs. Among the nine MAbs, three (#19A, #19B, #61) recognized only intact VLPs and six (#21, #25, #37, #41, #51B, #91) recognized both intact and disrupted VLPs by ELISA. Only one hybridoma cell line (#12) was generated from F₄₀₁-VLPs. MAb #12 only reacted with intact F₄₀₁-VLPs. Since MAb #12 was not reactive with wtVLPs, it was excluded from further studies.

Characterization of Monoclonal Antibodies Against Intact VLPs for their Topographical Location, Cross-Reactivity, and Hemagglutination Inhibition

MAbs were characterized for cross-reactivity, hemagglutination inhibition assay (HIA), and their specificity for conformational or sequential (linear) epitopes (Table A4). All 9 MAbs generated using N₃₉₉N₄₀₀F₄₀₁TDT₄₀₄-VLPs as immunogens were reactive with N₃₉₉N₄₀₀F₄₀₁TDT₄₀₄-VLPs, WT (Q399QYTDQ400) VLPs and F₄₀₁-VLPs (Table A2). These VLPs were antigenically similar, if not identical, to each other for the presentation of surface epitopes on VLPs. Eight MAbs (#19B, #21, #25, #37, #41, #51B, #61 and #91) from N₃₉₉N₄₀₀F₄₀₁TDT₄₀₄-VLPs were also reactive with VLPs of genotype 2 and 3 (Table A3).

TABLE A3
Cross-reactivities of generated MAbs against VLPs of three
genotypes of B19V

| | Monoclonal Antibody (MAb) | | | | | | | |
|---|---|---|---|---|---|---|---|---|
| Antigen | 19B | 21 | 25 | 37 | 41 | 51B | 61 | 91 |
| B19V Genotype 1 | 1.41 | 2.78 | 2.67 | 3.45 | 3.75 | 2.84 | 1.75 | 3.43 |
| B19V Genotype 2 | 0.90 | 2.39 | 2.54 | 3.11 | 3.29 | 2.59 | 1.35 | 3.09 |
| B19V Genotype 3 | 1.22 | 2.56 | 2.65 | 3.23 | 3.56 | 2.97 | 1.66 | 3.05 |

#19A and #12 were weakly positive, so not checked for cross-reactivity

TABLE A4
Summary on the characteristics of MAbs

| | Monoclonal Antibody (MAb) | | | | | | | | | |
|---|---|---|---|---|---|---|---|---|---|---|
| | | | 21 | 25 | 37 | 41 | 51B | | 91 | |
| | 19A | 19B | Both | Both | Both | Both | Both | 61 | Both | 12* |
| ELISA | Intact | Intact | Intact | Intact | Intact | Intact | Intact | Intact | Intact | Intact |
| (react | VLP | VLP | and | and | and | and | and | VLP | and | VLP |
| with) | only | only | dusrupted | dusrupted | dusrupted | dusrupted | dusrupted | only | dusrupted | only |
| IB* | Neg. | Neg. | Pos. | Pos. | Pos. | Pos. | Pos. | Neg. | Pos. | Weak |
| | | | | | | | | | | Pos. |
| HIA | Neg. | Pos. | Neg. | Pos. | Neg. | Neg. | Neg. | Pos. | Neg. | Neg. |

*Denatured condition
WT VLPs were employed for ELISA, IB, and HIA. For MAb-12*, however, F₄₀₁-VLPs were used for ELISA as well as IB

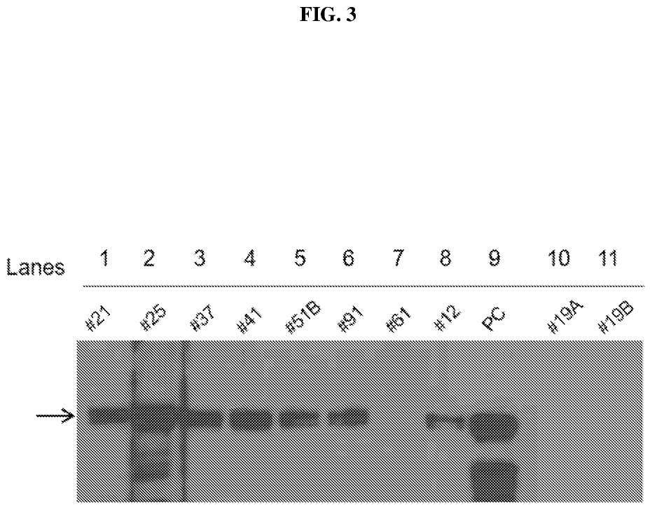

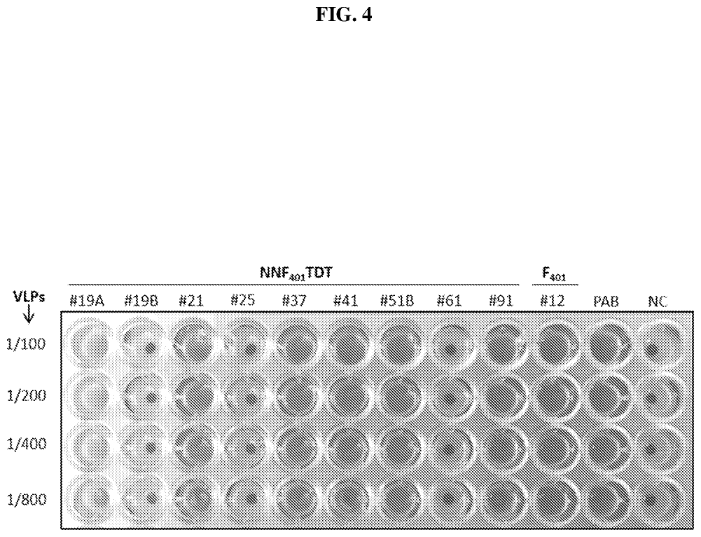

We did not test the cross-reactivity of the MAb #19A because of its low titer by ELISA. Three MAbs (#19A, #19B, #61) were determined to react only with the conformational epitopes on VLPs since they were positive with only intact VLPs by ELISA and negative by IB (FIG. 3) when tested with wtVLPs of genotype 1. Among these three, two (#19B and #61) inhibited the HA of red blood cells by wtVLPs, and #19A was negative in the HIA (FIG. 4). Six MAbs (#21, #25, #37, #41, #51B, #91) recognized sequential surface epitopes on VLPs. Except MAb #25, all other five MAbs (#21, #37, #41, #51B, #91) were not able to inhibit the binding of WT VLPs to RBCs (HIA negative; FIG. 4). MAb #25 recognized a sequential epitope (FIG. 3), and was HIA positive. All MAbs that recognized conformational epitopes by ELISA, did not react with N₃₉₉N₄₀₀F₄₀₁TDT₄₀₄-VP2 by IB as well, but all other MAbs reactive with denatured sequential epitopes of VLPs on ELISA also reacted with VP2 protein by IB (FIG. 3). The MAb #12 that was made against F₄₀₁-VLPs, was reactive with conformational epitopes only on F₄₀₁-VLPs, indicating the presence of a surface epitope that does not exist on wtVLPs. Naturally, MAb #12 did not inhibit the hemagglutinin by wtVLPs. This MAb, which was non-reactive with disruptive VLPs on ELISA, reacted weakly with VP2 proteins on immunoblot. None of the MAbs to VLPs reacted with HPV16 VLPs produced using the baculovirus expression system, thus demonstrating their specificity for VLPs.

According to our data from the electron microscopic analysis and ELISA using polyclonal antibodies to VLPs, both F₄₀₁-VLPs and N₃₉₉N₄₀₀F₄₀₁TDT₄₀₄-VLPs are morphologically and antigenically similar to wtVLPs, and share common epitopes.

In this study, nine distinct murine MAbs were generated against intact N₃₉₉N₄₀₀F₄₀₁TDT₄₀₄-VLPs. These MAbs were confirmed to also react with wtVLPs derived from the major capsid proteins of all three parvovirus B19 genotypes, suggesting the presence of shared epitopes on all three. All nine MAbs were specific to parvovirus B19, and were not reactive with the capsids of non-human erythroviruses (rhesus and pig-tailed parvoviruses; data not shown). Formation/exposure of these epitopes on the surface of these VLPs was consistent among all three genotypes. In the case of N₃₉₉N₄₀₀R₄₀₁TDT₄₀₄-VLPs, the surface sequential epitopes appeared to be highly immunogenic, resulting in the generation of multiple MAbs (six out of the nine MABs) against sequential epitopes. In some embodiments, conformational epitopes are recognized as immunodominant epitopes on VLPs. Of the six MAbs that were reactive with sequential surface epitopes, only one MAb (#25) inhibited the binding of wtVLPs to red blood cells. This suggests the existence of a sequential neutralizing epitope on VP2. Without wishing to be bound by theory, MAb #25 may have blocked or sterically hindered wtVLP from binding to the P-antigen on the red blood cells. In the murine system, when our mutated N₃₉₉N₄₀₀F₄₀₁TDT₄₀₄-VLPs were employed as immunogen, conformational, and potentially neutralizing, epitopes appeared to be immunodominant.

N₃₉₉N₄₀₀F₄₀₁TDT₄₀₄-VLPs have a four amino acid mutation in loop 4. N₃₉₉N₄₀₀F₄₀₁TDT₄₀₄-VLPs lack HA, and is a better immunogen than wtVLPs, at least in mice. N₃₉₉N₄₀₀F₄₀₁TDT₄₀₄- and wtVLPs, however, appeared to be antigenically identical. In contrast to N₃₉₉N₄₀₀F₄₀₁TDT₄₀₄-VLPs, F₄₀₁-VLPs generated only one hybridoma cell line (#12). The epitope of MAb #12 was absent on N₃₉₉N₄₀₀F₄₀₁TDT₄₀₄-VLPs as well as wtVLPs, and appeared to be artificially formed by the substitution of Y₄₀₁ to F₄₀₁. This mutation from Y₄₀₁ to F₄₀₁ in the recess region of 3-fold axes did not, however, seem to cause significant conformational or antigenic changes since polyclonal antibodies against wtVLPs and the nine MAbs to N₃₉₉N₄₀₀F₄₀₁TDT₄₀₄-VLPs were highly reactive with F₄₀₁-VLPs.

In conclusion, we generated nine MAbs reactive with intact wtVLP by employing mutated, hemagglutination-negative VLPs, which were found to be morphologically and antigenically indistinguishable from wtVLPs. These MAbs revealed that the folding of the VLPs with four mutated amino acids (N₃₉₉N₄₀₀F₄₀₁T₄₀₄) was exactly the same as that of wtVLPs. The surface epitopes on N₃₉₉N₄₀₀F₄₀₁TDT₄₀₄-VLPs were conserved across all three parvovirus B19 genotypes. The sole mutation from Y to F at the position 401 resulted in VLPs (F₄₀₁-VLPs) with artificial immunodominant epitopes. The results of our study clearly reveal that conformational, as well as sequential, surface epitopes exist on mutated VLPs that can be used to produce antibodies that inhibit hemagglutination of virions.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

In certain instances, sequences disclosed herein are included in publicly-available databases, such as GENBANK® and SWISSPROT. Unless otherwise indicated or apparent the references to such publicly-available databases are references to the most recent version of the database as of the filing date of this Application.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner Indeed, various modifications of the invention in addition to those described herein will become apparent to those skilled in the art from the foregoing description and the accompanying figures. Such modifications are intended to fall within the scope of the appended claims.

What is claimed is:

1. A VLP binding agent that specifically binds to a VLP, a parvovirus, an erythrovirus, or a parvovirus B19.

2. The VLP binding agent of claim 1, wherein the VLP is (a) a wtVLP, (b) an mVLP comprising a polypeptide comprising a VP2 polypeptide with at least one amino acid modification relative to a wild type VP2, or (c) both.

3. The VLP binding agent of claim 2, wherein the wild type VP2 has the amino acid sequence of SEQ ID NO: 1, and the at least one amino acid modification comprises a substitution at Y401, a substitution at Q399, a substitution at Q400, a substitution at Q404, a substitution at Q368, a substitution at Q369, a substitution at Y392, Y401F, Y401W, Y401A, Q368A, Q369A, Q368N, Q369N, Q399N, Q400N, Q404T, Y392A, Y392F, Q404N, Y401P, T₄₀₂A, D403A, Q404A, or combinations thereof.

4. The VLP binding agent of any of claims 2-3, wherein the at least one amino acid modification comprises one or more of Y401F, Y401W, Q368A, Q369A, Q399N, Q400N, or Q404T.

5. The VLP binding agent of any of claims 2-4, wherein VP2 polypeptide is construct A, construct B, construct D, or construct F.

6. The VLP binding agent of any of claims 1-5, wherein the VLP binding agent is an antibody, a monoclonal antibody, an antigen binding fragment, or an antibody fragment.

7. The VLP binding agent of any of claims 1-6, wherein the VLP binding agent comprises an amino acid sequence with (a) SEQ ID NOS:2-4 and 14-16, each with up to four conservative amino acid substitutions, (b) SEQ ID NOS:5-7 and 17-19, each with up to four conservative amino acid substitutions, (c) SEQ ID NOS:8-10 and 20-22, each with up to four conservative amino acid substitutions, or (d) SEQ ID NOS:11-13 and 23-25, each with up to four conservative amino acid substitutions.

8. The VLP binding agent of any of claims 1-7, wherein the VLP binding agent comprises an amino acid sequence with (a) SEQ ID NOS:2-4 and 14-16, (b) SEQ ID NOS:5-7 and 17-19, (c) SEQ ID NOS:8-10 and 20-22, or (d) SEQ ID NOS:11-13 and 23-25.

9. The VLP binding agent of any of claims 1-8, wherein the VLP binding agent comprises an amino acid sequence with (a) at least about 90% sequence identity to any of SEQ ID NOs:26-29; and/or (b) at least about 90% sequence identity to any of SEQ ID NOs:30-33.

10. The VLP binding agent of any of claims 1-9, wherein the VLP binding agent comprises an amino acid sequence with (a) at least one of SEQ ID NOs:26-29; and/or (b) at least one of SEQ ID NOs:30-33.

11. The VLP binding agent of any of claims 1-10, wherein the VLP binding agent comprises an amino acid sequence with (a) SEQ ID NOs:26 and 30, (b) SEQ ID NOs:27 and 31, (c) SEQ ID NOs:28 and 32, or (d) SEQ ID NOs:29 and 33.

12. The VLP binding agent of any of claims 1-11, wherein the VLP binding agent is detectably labeled.

13. A cell for producing the VLP binding agent of any of claims 1-12.

14. A method for making the VLP binding agent of any of claims 1-12 comprising (a) culturing the cell of claim 13, and (b) isolating the VLP binding agent.

15. A composition comprising the VLP binding agent of any of claims 1-12.

16. A pharmaceutical composition comprising the VLP binding agent of any of claims 1-12.

17. A polynucleotide comprising a polynucleotide that encodes the VLP binding agent of any of claims 1-12.

18. A method of detecting parvovirus, erythrovirus, parvovirus B19, or a VLP in a sample comprising contacting the sample with the VLP binding agent of any of claims 1-12, the composition of claim 15, or the pharmaceutical composition of claim 16.

19. The method of claim 18, wherein the VLP binding agent is detectably labeled.

20. The method of claim 18 or claim 19, wherein the label is selected from the group consisting of immunofluorescent label, chemiluminescent label, phosphorescent label, enzyme label, radiolabel, avidin/biotin, colloidal gold particles, colored particles and magnetic particles.

21. The method of any of claims 18-20, wherein the detecting is determined by radioimmunoassay, Western blot assay, cytometry, immunofluorescent assay, enzyme immunoassay, ELISA, immunoprecipitation assay, chemiluminescent assay, or immunohistochemical assay.

22. The method of any of claims 18-21, wherein the detecting is of (a) parvovirus, (b) parvovirus B19, (c) the wtVLP made from a VP2 polypeptide of SEQ ID NO:1 or (d) construct F.

23. A method for diagnosis in an animal with a parvovirus infection, an erythrovirus infection, or a parvovirus B19 infection, the method comprising
detecting whether parvovirus, erythrovirus, or parvovirus B19 is in a sample from the animal, comprising the method of detecting according to any of claims 18-22, and
diagnosing the animal with a parvovirus infection, an erythrovirus infection, or a parvovirus B19 infection, if the presence of parvovirus, erythrovirus, or parvovirus B19 in the sample is detected.

24. The method of claim 23, wherein the method is for diagnosis for a parvovirus infection or a parvovirus B19 infection.

25. The method of claim 23 or claim 24, wherein the animal is a mammal.

26. The method of any of claims 23-25, wherein the animal is a human.

27. A method for treating an animal for a parvovirus infection, a disease related to a parvovirus infection, an erythrovirus infection, a disease related to an erythrovirus infection, a parvovirus B19 infection, or a disease related to a parvovirus B19 infection, comprising one or more administrations of one or more compositions comprising one or more VLP binding agents of any of claims 1-12.

28. The method of claim 27, wherein at least one of the one or more compositions does not comprise an adjuvant.

29. The method of claim 27 or claim 28, wherein at least one of the one or more compositions further comprises a carrier or an adjuvant.

30. The method of any of claims 27-29, wherein at least one of the one or more the compositions comprises a pharmaceutical composition.

31. The method of any of claims 27-30, wherein at least one of the one or more administrations comprises parenteral administration, a mucosal administration, intravenous administration, subcutaneous administration, topical administration, intradermal administration, oral administration, sublingual administration, intranasal administration, or intramuscular administration.

32. The method of any of claims 27-31, wherein at least one of the one or more administrations comprises parenteral administration, intravenous administration, subcutaneous administration, or intramuscular administration.

33. The method of any of claims 27-32, wherein if there is more than one administration at least one composition used for at least one administration is different from the composition of at least one other administration.

34. The method of any of claims 27-33, wherein the animal is a human.

35. The method of any of claims 27-34, wherein the animal is in need of the treatment.

36. The method of any of claims 27-35, wherein the method is for treating an erythrovirus infection, a disease related to an erythrovirus infection, a parvovirus B19 infection, or a disease related to a parvovirus B19 infection.

37. The method of any of claims 27-36, wherein the method is for treating an erythrovirus infection, a parvovirus B19 infection, a disease related to an erythrovirus infection, a disease related to parvovirus B19 infection, hydrops fetalis intrauterine fetal death, erythema infectiosum (i.e., fifth disease), sickle cell anemia, Thalassemia, anemia, anemia induced by malaria, parvovirus B19-induced red cell aplasia (TRCA), chronic anemia, acute arthropathy, persistent arthropathy, aplastic crisis, arthritis, hepatitis, myocarditis, hepatosplenomegaly, systemic lupus erythematosus, meningiencephalitis, or fibromyalgia.

38. The method of any of claims 27-37, wherein the method induces an immune response, is a therapeutic treatment, or is a combination thereof.

39. A method for inducing an immune response in an animal, comprising one or more administrations of one or more compositions comprising one or more VLP binding agents of any of claims 1-12.

40. The method of claim 39, wherein at least one of the one or more compositions does not comprise an adjuvant.

41. The method of claim 39 or claim 40, wherein at least one of the one or more compositions further comprises a carrier or an adjuvant.

42. The method of any of claims 39-41, wherein at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, adjuplex vaccine adjuvant, MF59, titermax, or combinations thereof.

43. The method of any of claims 39-42, wherein at least one of the one or more the compositions comprises a pharmaceutical composition.

44. The method of any of claims 39-43, wherein at least one of the one or more administrations comprises parenteral administration, a mucosal administration, intravenous administration, subcutaneous administration, topical administration, intradermal administration, oral administration, sublingual administration, intranasal administration, or intramuscular administration.

45. The method of any of claims 39-44, wherein if there is more than one administration at least one composition used for at least one administration is different from the composition of at least one other administration.

46. The method of any of 39-45, wherein the animal is a human.

47. The method of any of claims 39-46, wherein the animal is in need of the treatment.

48. The method of any of claims 39-47, wherein the method is for treating an erythrovirus infection, a disease related to an erythrovirus infection, a parvovirus B19 infection, or a disease related to a parvovirus B19 infection.

49. The method of any of claims 39-48, wherein the method is for treating an erythrovirus infection, a parvovirus B19 infection, a disease related to an erythrovirus infection, a disease related to parvovirus B19 infection, hydrops fetalis intrauterine fetal death, erythema infectiosum (i.e., fifth disease), sickle cell anemia, Thalassemia, anemia, anemia induced by malaria, parvovirus B19-induced red cell aplasia (TRCA), chronic anemia, acute arthropathy, persistent arthropathy, aplastic crisis, arthritis, hepatitis, myocarditis, hepatosplenomegaly, systemic lupus erythematosus, meningiencephalitis, or fibromyalgia.

50. The method of any of claims 39-49, wherein the method induces an immune response, is a therapeutic treatment, or is a combination thereof.

51. A method for treating an animal for a parvovirus infection, a disease related to a parvovirus infection, an erythrovirus infection, a disease related to an erythrovirus infection, a parvovirus B19 infection, or a disease related to a parvovirus B19 infection, comprising
detecting whether parvovirus, erythrovirus, or parvovirus B19 is in a sample from the animal, comprising the method of detecting according to any of claims 18-22, and
administering one or more administrations of one or more compositions comprising one or more of an mVLP, a VLP binding agent of any of claims 1-12, or an antibiotic, if the presence of parvovirus, erythrovirus, or parvovirus B19 in the sample is detected.

52. The method of claim 51, wherein the antibiotic comprises ampicillin, a cephalexin, or a flouroquinolone, or combinations thereof.

53. The method of claim 51 or claim 52, wherein (a) the mVLP comprises a VP2 polypeptide with at least one amino acid modification relative to a wild type VP2 and (b) the wild type VP2 has the amino acid sequence of SEQ ID NO: 1.

54. The method of any of claims 51-53, wherein (a) the mVLP comprises a VP2 polypeptide with at least one amino acid modification relative to a wild type VP2, (b) the wild type VP2 has the amino acid sequence of SEQ ID NO: 1, (c) the at least one amino acid modification (1) comprises (i) Y401F and (ii) Q399N or Q404T, (2) is Y401F, (3) is Q368A and Q369A, (4) is Q399N, Q400N, and Q404T, or (5) is Y392A, and (d) the VP2 polypeptide is not construct J.

55. The method of any of claims 51-54, wherein the VP2 polypeptide sequence has at least about 90% identity to SEQ ID NO: 1.

56. The method of any of claims 51-55, wherein the VP2 polypeptide sequence has at least about 95% identity to SEQ ID NO: 1.

57. The method of any of claims 51-56, wherein the at least one amino acid modification comprises (a) Y401F and (b) Q399N or Q404T.

58. The method of any of claims 51-57, wherein the VP2 polypeptide is selected from the group consisting of construct A, construct D, construct F, construct G, and construct H.

59. The method of any of claims 51-58, wherein the VP2 polypeptide is Construct F.

60. The method of any of claims 51-59, wherein the mVLP comprises a VP2 that has at least one amino acid modification (1) comprising (a) Y401F and (b) Q399N or Q404T or (2) is Y401F, and the VP2 polypeptide is not construct J.

61. The method of any of claims 51-60, wherein at least one of the one or more compositions does not comprise an adjuvant.

62. The method of any of claims 51-61, wherein at least one of the one or more compositions further comprises a carrier or an adjuvant.

63. The method of any of claims 51-62, wherein at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, adjuplex vaccine adjuvant, MF59, titermax, or combinations thereof.

64. The method of any of claims 51-63, wherein at least one of the one or more the compositions comprises a pharmaceutical composition.

65. The method of any of claims 51-64, wherein at least one of the one or more administrations comprises parenteral administration, a mucosal administration, intravenous administration, subcutaneous administration, topical administration, intradermal administration, oral administration, sublingual administration, intranasal administration, or intramuscular administration.

66. The method of any of claims 51-65, wherein if there is more than one administration at least one composition used for at least one administration is different from the composition of at least one other administration.

67. The method of any of claims 51-66, wherein the mVLP of at least one of the one or more compositions is administered to the animal in an amount of from about 0.01 mg of mVLP/kg animal body weight to about 15 mg of mVLP/kg animal body weight.

68. The method of any of claims 51-67, wherein the animal is a human.

69. The method of any of claims 51-68, wherein the animal is in need of the treatment.

70. The method of any of claims 51-69, wherein the method is for treating an erythrovirus infection, a disease related to an erythrovirus infection, a parvovirus B19 infection, or a disease related to a parvovirus B19 infection.

71. The method of any of claims 51-70, wherein the method is for treating an erythrovirus infection, a parvovirus B19 infection, a disease related to an erythrovirus infection, a disease related to parvovirus B19 infection, hydrops fetalis intrauterine fetal death, erythema infectiosum (i.e., fifth disease), sickle cell anemia, Thalassemia, anemia, anemia induced by malaria, parvovirus B19-induced red cell aplasia (TRCA), chronic anemia, acute arthropathy, persistent arthropathy, aplastic crisis, arthritis, hepatitis, myocarditis, hepatosplenomegaly, systemic lupus erythematosus, meningiencephalitis, or fibromyalgia.

72. The method of any of claims 51-71, wherein the method induces an immune response, is a therapeutic treatment, or is a combination thereof.

73. A method for inducing an immune response in an animal comprising
detecting whether parvovirus, erythrovirus, or parvovirus B19 is in a sample from the animal, comprising the method of detecting according to any of claims 18-22, and
administering one or more administrations of one or more compositions comprising one or more of an mVLP, a VLP binding agent of any of claims 1-12, or an antibiotic, if the presence of parvovirus, erythrovirus, or parvovirus B19 in the sample is detected.

74. The method of claim 73, wherein the antibiotic comprises ampicillin, a cephalexin, or a flouroquinolone, or combinations thereof.

75. The method of claim 73 or claim 74, wherein (a) the mVLP comprises a VP2 polypeptide with at least one amino acid modification relative to a wild type VP2 and (b) the wild type VP2 has the amino acid sequence of SEQ ID NO: 1.

76. The method of any of claims 73-75, wherein (a) the mVLP comprises a VP2 polypeptide with at least one amino acid modification relative to a wild type VP2, (b) the wild type VP2 has the amino acid sequence of SEQ ID NO: 1, (c) the at least one amino acid modification (1) comprises (i) Y401F and (ii) Q399N or Q404T, (2) is Y401F, (3) is Q368A and Q369A, (4) is Q399N, Q400N, and Q404T, or (5) is Y392A, and (d) the VP2 polypeptide is not construct J.

77. The method of any of claims 73-76, wherein the VP2 polypeptide sequence has at least about 90% identity to SEQ ID NO: 1.

78. The method of any of claims 73-77, wherein the VP2 polypeptide sequence has at least about 95% identity to SEQ ID NO: 1.

79. The method of any of claims 73-78, wherein the at least one amino acid modification comprises (a) Y401F and (b) Q399N or Q404T.

80. The method of any of claims 73-79, wherein the VP2 polypeptide is selected from the group consisting of construct A, construct D, construct F, construct G, and construct H.

81. The method of any of claims 73-80, wherein the VP2 polypeptide is Construct F.

82. The method of any of claims 73-81, wherein at least one of the one or more compositions does not comprise an adjuvant.

83. The method of any of claims 73-82, wherein at least one of the one or more compositions further comprises a carrier or an adjuvant.

84. The method of any of claims 73-83, wherein if there is more than one administration at least one composition used for at least one administration is different from the composition of at least one other administration.

85. The method of any of claims 73-84, wherein the animal is a human.

86. The method of any of claims 73-85, wherein at least one of the one or more compositions does not comprise an adjuvant.

87. The method of any of claims 73-86, wherein at least one of the one or more compositions further comprises a carrier or an adjuvant.

88. The method of any of claims 73-87, wherein at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, adjuplex vaccine adjuvant, MF59, titermax, or combinations thereof.

89. The method of any of claims 73-88, wherein at least one of the one or more the compositions comprises a pharmaceutical composition.

90. The method of any of claims 73-89, wherein at least one of the one or more administrations comprises parenteral administration, a mucosal administration, intravenous administration, subcutaneous administration, topical administration, intradermal administration, oral administration, sublingual administration, intranasal administration, or intramuscular administration.

91. A method for providing an animal with a VLP binding agent comprising one or more administrations of one or more compositions comprising the VLP binding agent of any of claims 1-12, wherein the compositions may be the same or different if there is more than one administration.

92. The method of claim 91, wherein at least one of the one or more compositions does not comprise an adjuvant.

93. The method of claim 91 or claim 92, wherein at least one of the one or more compositions further comprises a carrier or an adjuvant.

94. The method of any of claims 91-93, wherein at least one of the one or more compositions further comprises squalene, IL-2, RIBI adjuvant system, QS21, GM-CSF, alum hydro gel, monophosphoryl lipid A, trehalose dimycolate, Toll-like receptor ligands, Toll-like receptor agonists, CpG oligodeoxynucleotides, cell wall skeleton, adjuplex vaccine adjuvant, MF59, titermax, or combinations thereof.

95. The method of any of claims 91-94, wherein at least one of the one or more compositions comprises the composition of claim 15 or the pharmaceutical composition of claim 16.

96. The method of any of claims 91-95, wherein at least one of the one or more administrations comprises parenteral administration, a mucosal administration, intravenous administration, subcutaneous administration, topical administration, intradermal administration, oral administration, sublingual administration, intranasal administration, or intramuscular administration.

97. The method of any of claims 91-96, wherein if there is more than one administration at least one composition used for at least one administration is different from the composition of at least one other administration.

98. The method of any of claims 91-97, wherein the animal is a human.

99. The method of any of claims 91-98, wherein at least one of the one or more compositions further comprises a VLP is any of claim 2-5, 51-60, or 75-81.

100. The method of any of claims 91-99, wherein at least one of the one or more compositions further comprises an antibiotic.

101. The method of any of claims 91-100, wherein at least one of the one or more compositions further comprises an antibiotic and the antibiotic comprises ampicillin, a cephalexin, or a flouroquinolone, or combinations thereof.
